(19) United States
(12) Patent Application Publication
(10) Pub. No.: US 2018/0064829 A1
(43) Pub. Date: Mar. 8, 2018

(54) FLUORESCENT BISPHOSPHONATE ANALOGS

(71) Applicant: University of Southern California, Los Angeles, CA (US)

(72) Inventors: Charles E. McKenna, Pacific Palisades, CA (US); Boris A. Kashemirov, Los Angeles, CA (US); Shuting Sun, Los Angeles, CA (US); Feng Ni, Los Angeles, CA (US)

(73) Assignee: University of Southern California, Los Angeles, CA (US)

(21) Appl. No.: 15/563,318

(22) PCT Filed: Apr. 2, 2016
(86) PCT No.: PCT/US2016/025795

§ 371 Date: Sep. 29, 2017

Related U.S. Application Data
(60) Provisional application No. 62/142,437, filed on Apr. 2, 2015.
(60) Provisional application No. 62/142,428, filed on Apr. 2, 2015.

(51) Int. Cl. A61K 49/00; C07F 9/58; C07F 9/6506; C07F 9/6558; C07F 9/6561

(52) U.S. Cl. CPC A61K 49/0052; A61K 49/0043; A61K 49/0032; C07F 9/6561; C07F 9/65061; C07F 9/65583; C07F 9/582

(57) ABSTRACT

Fluorescent probes based on N-heterocyclic bisphosphonates or their phosphonocarboxylate analogues are provided. The probes have variable spectroscopic properties, bone mineral binding affinities, and pharmacological activities. Methods for preparing the probes include the use of two complementary linking strategies, one involving an amino group and the other involving a chloride group as a precursor to an amino group. In other versions, bifunctional N-heterocyclic bisphosphonates are provided having an amino group and an azido group as linking moieties. In some versions, the linking chemistry allows attachment of a wide selection of fluorescent dyes in the visible to near-infrared range to any of three clinically important heterocyclic bisphosphonates.

### CROSS-REFERENCE TO RELATED APPLICATIONS

This application claims the benefit of Provisional Patent Application Nos. 62/142,428, filed on Apr. 2, 2015, and 62/142,437, filed on Apr. 2, 2015, which are incorporated by reference herein.

### BACKGROUND

### Field of the Invention

The invention relates to bisphosphonates and their carboxyphosphonate analogues, and uses thereof.

### Related Art

Bisphosphonates (BPs) are therapeutic agents for the treatment of bone disorders such as osteoporosis and Paget's disease and have use in cancer treatment [1]. Farnesyl pyrophosphate synthase (FPPS) is known to be the primary intracellular enzymatic target for the antiresorptive activity of nitrogen-containing bisphosphonates (N-BPs), but details of the pharmacology, such as skeletal distribution and cellular uptake, remain to be fully elucidated [1b]. High-dose usage of N-BPs in some cancer patients has been associated with a side effect, known as osteonecrosis-of-the-jaw (ONJ) [2], however, its etiology and mechanism remain unclear [3]. These unsolved questions about bone structure, function and response to anti-resorptive drugs point to a need for new imaging agents able to mimic N-BPs, both with respect to their affinity for bone mineral and their cellular effects.

The bisphosphonate P—C—P bond of BPs mimics the P—O—P bond of the naturally-occurring bone metabolism mediator, inorganic pyrophosphate. Thus the BPs also retain strong binding affinity to hydroxyapatite (HAP), the major inorganic material found in bone, and exhibit exceptional stability against both chemical and biological degradation. This specific bone-targeting property of BPs also makes them an ideal carrier to introduce desired drugs or macromolecules to bone in drug delivery studies [4]. In addition, due to their strong and selective affinity to HAP, modified BPs with an appropriate imaging label can be used as molecular indicators for mapping breast cancer microcalcification, calcium urolithiasis, and atherosclerosis [5]. Thus, fluorescent probes of bisphosphonates are of intense interest as biological probes in imaging studies.

Compared with radioactive isotope-labeled imaging probes, fluorescent probes can be highly sensitive probes that may offer lower potential long-term toxicity [6]. Near-infrared (NIR) imaging probes exhibiting emission wavelengths between 700-1000 nm are ideal for in vivo imaging because tissue autofluorescence is minimized in this optical window [7]. A refined NIR Fluorescence-Assisted Resection and Exploration (FLARE) imaging system was recently introduced and utilized in a first-in-human testing in women undergoing sentinel lymph node mapping for breast cancer [8]. The successful clinical translation of this system may also offer the advantages of NIR imaging for image-guided oncologic surgery [9] and exemplifies the great potential of NIR imaging in disease prognosis and monitoring treatment effects in real time [10].

Early generation N-BPs (alendronate [11] and pamidronate [5a,12]) conjugated to Alexa Fluor 488™ and carboxyfluorescein were reported previously, but no cellular activity was reported [11]. Similarly, near-IR analogues of pamidronate, including Pain78, Pam800, and commercialized OsteoSenseTm680 EX and 750 EX, have also been visualized in vitro and in vivo, but their pharmacological activity has not been documented. Their synthetic chemistry may suffer from either low yields or a complicated purification procedure [5a,12]. In general, alendronate/pamidronate were conjugated to the activated carboxyl form of a dye, converting the terminal amino group of the N-BP to an amido linkage, thereby drastically modifying a key pharmacophore in the original N-BP. Direct acylation of the amino nitrogen in N-BPs by an activated fluorescent label is not readily applicable to the more potent modern heterocyclic N-BP drugs, such as risedronate (RIS, 1a of Scheme 1), zoledronate (ZOL, 1d of Scheme 1) and minodronate (MIN, 1e of Scheme 1), which lack a primary amino group. Thus, new ways of linking N-BPs to tags and molecules of interest are desirable.

### SUMMARY

In one aspect, new methods to link fluorescent dyes and heterocyclic bisphosphonate drugs, such as zoledronate, risedronate and minodronate, are provided. This enabled synthesis of a toolkit combining all heterocyclic bisphosphonate drugs used in the clinic, or their phosphonocarboxylate analogues, with fluorescent dyes via a linker. The toolkit includes the idea of being able to combine any modern heterocyclic bisphosphonate drug with any suitably activated visible or near-IR dye using a linker strategy, to obtain a range of bone affinities, fluorescent properties, and presence or absence of biological activity. The toolkit creation incorporates a previously disclosed linker method but introduces novel methods that makes it possible to extend linking to additional bisphosphonate drugs and has other advantages.

Nitrogen-containing bisphosphonates include clinically relevant N-BPs such as (1-hydroxy-2-pyridin-3-ylethane-1,1-diyl)bis(phosphonic acid) 1, [hydroxy(1H-imidazol-1-yl)methylene]bis(phosphonic acid), {1-hydroxy-3-[methyl(pentyl)amino]propane-1,1-diyl}bis(phosphonic acid), (3-amino-1-hydroxypropane-1,1-diyl)bis(phosphonic acid), and (4-amino-1-hydroxybutane-1,1-diyl)bis(phosphonic acid).

In one aspect, a toolkit for use in bone tissue is provided. The toolkit includes a plurality of N-heterocyclic bisphosphonates, or phosphonocarboxylate analogues thereof, linked to imaging tags, where the imaging tag-linked N-heterocyclic bisphosphonates, or phosphonocarboxylate analogues thereof, can exhibit preselected characteristics such as variable physical, optical (see Table 1) and biological characteristics. Optical properties can include, but are not limited to, light absorption and emission frequencies, which could range between 200 and 900 nm. Physical properties can include, but are not limited to, bone mineral binding affinities, which can vary from high to low. Biological activities can include, but are not limited to, antiprenylation activities, which can vary from high to negligible.

In some embodiments, the toolkit includes the imaging tag-linked N-heterocyclic bisphosphonates, or the phosphonocarboxylate analogues thereof, included in Table 1 and selected from the group consisting of 5 (6)-FAM-RIS (7a1), 5-FAM-RIS (7a2), 6-FAM-RIS (7a3), 5 (6)-RhR-RIS (7a4), 5 (6)-ROX-RIS (7a5), AF647-RIS (7a6), 5 (6)-FAM-RISPC (7b1), 5 (6)-RhR-RISPC (7b2), 5 (6)-ROX-RISPC (7b3), AF647-RISPC (7b4), 5 (6)-FAM-dRIS (7c1), 5 (6)-RhR-dRIS (7c2), 5-FAM-ZOL (7d1), 6-FAM-ZOL (7d2), AF647-ZOL (7d3), 800 CW-ZOL (7d4), Sulfo-Cy5-ZOL (7d5), 5-FAM-MIN (7e1), 6-FAM-MIN (7e2), 5-FAM-MINPC (7f1), and 6-FAM-MINPC (712).

In another aspect, a method for analyzing bone, bone metabolism, bone interaction with drugs, BP dosing to bone, or BP distribution within bone, or any combination thereof, is provided. The method includes exposing a bone to the imaging tag-linked N-heterocyclic bisphosphonates, or the phosphonocarboxylate analogues thereof, or pharmaceutically acceptable salts thereof, of the toolkit. In the method, the bone can be inside of a subject's body, or in other cases, the bone can be removed from a subject and be external to the subject's body.

In a further aspect, a method for treating a bone or bone-related disease in a subject in need thereof, is provided. The method includes treating the subject with one or more of the imaging tag-linked N-heterocyclic bisphosphonates, or the phosphonocarboxylate analogues thereof, or pharmaceutically acceptable salts thereof, of the toolkit, and visualizing the one or more bisphosphonates, or the phosphonocarboxylate analogues thereof, in the subject by in situ fluorescence. The disease can be, but is not limited to, osteoporosis, Paget's disease, osteonecrosis of the jaw, or any bone related cancer such as, for example, metastatic cancer to bone or multiple myeloma, or the like.

In a further aspect, a composition is provided for treating a bone or bone-related disease in a subject in need thereof that includes one or more of the imaging tag-linked N-heterocyclic bisphosphonates, or the phosphonocarboxylate analogues thereof, or pharmaceutically acceptable salts thereof, of the toolkit, and visualizing the one or more bisphosphonates, or the phosphonocarboxylate analogues thereof, in the subject by in situ fluorescence.

In another aspect, a method of preparing a kit (toolkit) for bone imaging is provided. The method includes combining a plurality of N-heterocyclic bisphosphonates, or phosphonocarboxylate analogues thereof, with activated imaging tags so as to form a toolkit of imaging tag-linked N-heterocyclic bisphosphonates, or phosphonocarboxylate analogues thereof, or pharmaceutically acceptable salts thereof, that exhibit a range of preselected physical, optical and biological characteristics, where the imaging tags are linked to the N-heterocyclic bisphosphonates, or the phosphonocarboxylate analogues thereof, by reacting activated imaging tags to ammonolized halogen-containing N-heterocyclic bisphosphonates, or phosphonocarboxylate analogues thereof, and to amino-group containing N-heterocyclic bisphosphonates, or phosphonocarboxylate analogues thereof.

In some embodiments, imaging tags can be attached to halogen-containing linker-N-heterocyclic bisphosphonate or phosphonocarboxylate analogue conjugates by conversion of the alkyl halide moiety to the alkyl amine by reaction with ammonia, followed by reaction with an imaging tag containing an activated carboxylate group known to those of skill in the art, such as a succinimidyl ester. Alternatively, an activating reagent known to those of skill in the art, such as dicyclohexyldicarbonimide, may be used with the unmodified carboxylate group.

In some embodiments, the imaging tag-linked N-heterocyclic bisphosphonates, or the phosphonocarboxylate analogues thereof, are included in Table 1 and selected from the group consisting of 5 (6)-FAM-RIS (7a1), 5-FAM-RIS (7a2), 6-FAM-RIS (7a3), 5 (6)-RhR-RIS (7a4), 5 (6)-ROX-RIS (7a5), AF647-RIS (7a6), 5 (6)-FAM-RISPC (7b1), 5 (6)-RhR-RISPC (7b2), 5 (6)-ROX-RISPC (7b3), AF647-RISPC (7b4), 5 (6)-FAM-dRIS (7c1), 5 (6)-RhR-dRIS (7c2), 5-FAM-ZOL (7d1), 6-FAM-ZOL (7d2), AF647-ZOL (7d3), 800 CW-ZOL (7d4), Sulfo-Cy5-ZOL (7d5), 5-FAM-MIN (7e1), 6-FAM-MIN (7e2), 5-FAM-MINPC (7f1), and 6-FAM-MINPC (7f2).

In a further aspect, a method of preparing a modified N-heterocyclic bisphosphonate, or a phosphonocarboxylate analogue thereof, is provided. The method includes: a) reacting an N-heterocyclic bisphosphonate, or a phosphonocarboxylate analogue thereof, with a haloepoxide to produce a halogen-containing N-heterocyclic bisphosphonate, or a phosphonocarboxylate analogue thereof, and b) converting the halogen-containing N-heterocyclic bisphosphonate, or a phosphonocarboxylate analogue thereof, to an amino group-containing N-heterocyclic bisphosphonate, or a phosphonocarboxylate analogue thereof.

In some embodiments of the method: a) the N-heterocyclic bisphosphonate, or the phosphonocarboxylate analogue thereof, has the formula:

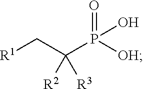

b) the halogen-containing N-heterocyclic bisphosphonate, or the phosphonocarboxylate analogue thereof, has the formula:

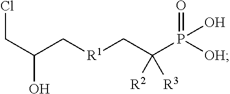

or c) the amino group-containing N-heterocyclic bisphosphonate, or the phosphonocarboxylate analogue thereof, has the formula:

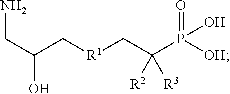

where R¹ is an imidazole, a pyridine, or an imidazo[3,2-a]pyridine, R² is H or OH; and R³ is P(O)(OH)₂ or C(O)OH.

In particular embodiments of the method:

a) the imidazole is

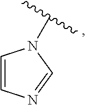 [SMILES: *n1ccnc1]

or an analogue thereof;
b) the pyridine is

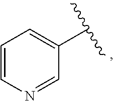 [SMILES: *c1cccnc1]

or an analogue thereof;
and c) the imidazo[3,2-a]pyridine is

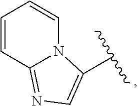 [SMILES: *c1cnc2ccccn12]

or an analogue thereof.

The analogue can be, but is not limited to, an alkyl substituted and/or halogen substituted embodiment of the imidazole, pyridine, or imidazo[3,2-a]pyridine.

In some embodiments, the haloepoxide is epichlorohydrin.

The analogue can be, but is not limited to, an alkyl substituted and/or halogen substituted embodiment of the imidazole, pyridine, and imidazo[3,2-a]pyridine.

In some embodiments, the method further includes reacting an amino group of the amino group-containing N-heterocyclic bisphosphonate, or the phosphonocarboxylate analogue thereof, with an imaging tag such as, but not limited to, 5 (6)-Carboxyfluorescein, Rhodamine Red X, X-Rhodamine, Alexa Fluor 647, IRDye 800 CW, or Sulfo-Cy5, all in an activated form. In some embodiments, the imaging tag comprises a fluorescent dye, which can be an activated succinimidyl ester-containing fluorescent dye for reaction with the amino group.

In some embodiments of the method, the reacting of the amino group with the imaging tag produces an N-heterocyclic bisphosphonate, or the phosphonocarboxylate analogue thereof, of the formula:

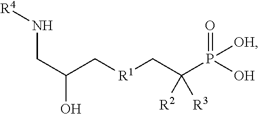

where Fe is an imidazole, a pyridine, or an imidazo[3,2-a]pyridine, R² is H or OH; R³ is P(O)(OH)₂ or C(O)OH; and R⁴ comprises a fluorescent dye. In particular embodiments, the fluorescent dye can be 5 (6)-Carboxyfluorescein, Rhodamine Red X, X-Rhodamine, Alexa Fluor 647, IRDye 800 CW, or Sulfo-Cy5, all in activated form.

In some embodiments of the method, the yield of the fluorescently tagged amino group-containing N-heterocyclic bisphosphonate, or a phosphonocarboxylate analogue thereof, is about 50%-77%.

In another aspect, a method of preparing a modified N-heterocyclic bisphosphonate or a phosphonocarboxylate analogue thereof, is provided. The method includes:

a) reacting an epoxide having a protected amino group with an ester-protected N-heterocylic bisphosphonate, or a phosphonocarboxylate analogue thereof, to produce a protected amino group- and hydroxyl group-containing ester-protected N-heterocylic bisphosphonate, or a phosphonocarboxylate analogue thereof;

b) reacting the protected amino group- and hydroxyl group-containing ester-protected N-heterocylic bisphosphonate, or the phosphonocarboxylate analogue thereof, with a sulfonyl halide to produce a sulfonylated and protected amino group-containing ester-protected N-heterocylic bisphosphonate, or a phosphonocarboxylate analogue thereof;

c) reacting the sulfonylated and protected amino group-containing ester-protected N-heterocylic bisphosphonate, or the phosphonocarboxylate analogue thereof, with an azide to produce a protected amino group- and azido-containing ester-protected N-heterocylic bisphosphonate, or a phosphonocarboxylate analogue thereof; and

d) deprotecting the protected amino group- and azido-containing ester-protected N-heterocylic bisphosphonate, or the phosphonocarboxylate analogue thereof, to produce an N-heterocylic bisphosphonate comprising an azido group and an amino group, or a phosphonocarboxylate analogue thereof.

In some embodiments of the method:

a) the ester-protected N-heterocylic bisphosphonate, or phosphonocarboxylate analogue thereof, has the formula:

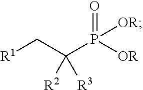

b) the protected amino group- and hydroxyl group-containing ester-protected N-heterocylic bisphosphonate, or phosphonocarboxylate analogue thereof, has the formula:

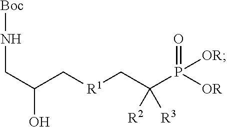

c) the sulfonylated and protected amino group-containing ester-protected N-heterocylic bisphosphonate, or phosphonocarboxylate analogue thereof, has the formula:

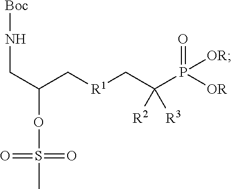

d) the protected amino group- and azido-containing ester-protected N-heterocylic bisphosphonate, or phosphonocarboxylate analogue thereof, has the formula:

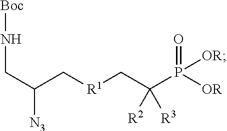

or e) the N-heterocylic bisphosphonate comprising an azido group and an amino group, or phosphonocarboxylate analogue thereof, has the formula:

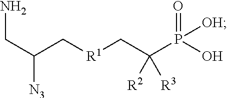

wherein R=Me, Et, Pr or iPr; R¹ is an imidazole, a pyridine, or an imidazo[3,2-a]pyridine; R² is H or OH; and R³ is P(O)(OH)₂ or C(O)OH.

In particular embodiments of the method:

a) the imidazole is

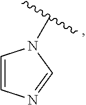 [SMILES: *n1ccnc1]

or an analogue thereof;
b) the pyridine is

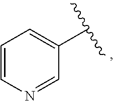 [SMILES: *c1cccnc1]

or an analogue thereof;
and c) the imidazo[3,2-a]pyridine is

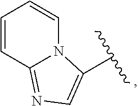 [SMILES: *c1cnc2ccccn12]

or an analogue thereof.

The analogue can be, but is not limited to, an alkyl substituted and/or halogen substituted embodiment of the imidazole, pyridine, or imidazo[3,2-a]pyridine.

In particular embodiments of the method, the epoxide has the formula

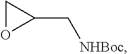 [SMILES: CC(C)(C)OC(=O)NCC1CO1]

the sulfonyl halide is methanesulfonyl chloride, or the azide is NaN₃, or any combination thereof.

In some embodiments of the method, the method further includes reacting the azido group or the amino group, or each independently, to a substance of the group consisting of an: a) imaging tag such as, but not limited to, a fluorescent dye such as (6)-Carboxyfluorescein, Rhodamine Red X, X-Rhodamine, Alexa Fluor 647, IRDye 800 CW, and Sulfo-Cy5, or a fluorescence quencher such as Dabcyl, BHQ-1, BHQ-2, BHQ-3, QSY 7, and QSY 9; b) a drug such as, but not limited to, compounds effective for treatment of disorders of bone, such as an N-bisphosphosphonate, compounds effective for the treatment of cancer, or for compounds effective for the treatment of diseases caused by microbial infections, such as an antibiotic or antiviral drug; c) a protein; d) a peptide; e) an oligonucleotide; f) a nanoparticle; and g) a polymer. In particular embodiments, the substance includes an activated succinimidyl ester for reaction with the amino group, or comprises an alkyne for reaction with the azide group.

In some embodiments of the method, the yield of the N-heterocylic bisphosphonate comprising an azido group and an amino group, or a phosphonocarboxylate analogue thereof, is about 28%.

In another aspect, a bifunctional N-heterocylic bisphosphonate comprising an azido group and an amino group, or a phosphonocarboxylate analogue thereof, is provided. In some embodiments, the compound has the formula:

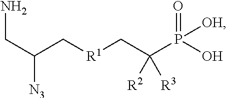

wherein R=Me, Et, Pr or iPr; R¹ is an imidazole, a pyridine, or an imidazo[3,2-a]pyridine; R² is H or OH; and R³ is P(O)(OH)₂ or C(O)OH.

In particular embodiments:

a) the imidazole is

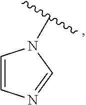 [SMILES: *n1ccnc1]

or an analogue thereof;
b) the pyridine is

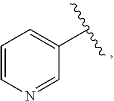 [SMILES: *c1cccnc1]

or an analogue thereof;
and c) the imidazo[3,2-a]pyridine is

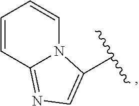 [SMILES: *c1cnc2ccccn12]

or an analogue thereof.

The analogue can be, but is not limited to, an alkyl substituted and/or halogen substituted embodiment of the imidazole, pyridine, or imidazo[3,2-a]pyridine.

In a further aspect, a composition comprising an N-heterocylic bisphosphonate, or a phosphonocarboxylate analogue thereof, attached to a substance, is provided. In some embodiments, the composition has the formula:

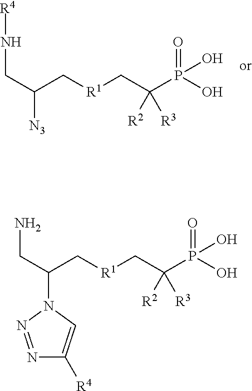

wherein R¹ is an imidazole, a pyridine, or an imidazo[3,2-a]pyridine; R² is H or OH; R³ is P(O)(OH)₂ or C(O)OH, and R⁴ comprises a substance selected from the group consisting of an imaging tag, a drug, a protein, a peptide, an oligonucleotide, a nanoparticle and a polymer.

In particular embodiments:

a) the imidazole is

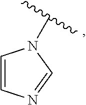 [SMILES: *n1ccnc1]

or an analogue thereof;
b) the pyridine is

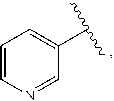 [SMILES: *c1cccnc1]

or an analogue thereof;
and c) the imidazo[3,2-a]pyridine is

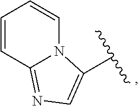 [SMILES: *c1cnc2ccccn12]

or an analogue thereof.

The analogue can be, but is not limited to, an alkyl substituted and/or halogen substituted embodiment of the imidazole, pyridine, or imidazo[3,2-a]pyridine.

In some embodiments, the substance is a fluorescent dye or a fluorescence quencher, and in particular embodiments, the fluorescent dye can be 5 (6)-Carboxyfluorescein, Rhodamine Red X, X-Rhodamine, Alexa Fluor 647, IRDye 800 CW, or Sulfo-Cy5; and the fluorescence quencher can be Dabcyl, BHQ-1, BHQ-2, BHQ-3, QSY 7, or QSY 9.

In another embodiment, a method of preparing a modified N-heterocyclic bisphosphonate, or a phosphonocarboxylate analogue thereof, is provided. The method includes reacting an N-heterocyclic bisphosphonate, or a phosphonocarboxylate analogue thereof, with an azido epoxide to produce an azido-containing N-heterocyclic bisphosphonate, or a phosphonocarboxylate analogue thereof.

In some embodiments of the method, the azido epoxide is

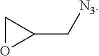

In some embodiments of the method, a) the method further includes reacting an azido group of the azido-containing N-heterocyclic bisphosphonate, or the phosphonocarboxylate analogue thereof, to a substance of the group consisting of an imaging tag, a drug, a protein, a peptide, an oligonucleotide, a nanoparticle and a polymer, wherein the substance contains an alkyne group for reaction with the azido group; b) the method can be used to prepare an imaging probe by reducing the azido group of the azido-containing N-heterocyclic bisphosphonate-linker compound to a primary amino group, then reacting the primary amino group with an activated carboxylate ester group of an imaging tag; c) the imaging tag can be a fluorescent dye; d) the N-heterocyclic bisphosphonate can be any of the N-heterocyclic bisphosphonates described for use with other methods and embodiments of any other aspect of the invention.

In any of the methods, compositions, treatments, analysis methods and toolkits described above, an imaging tag can be any fluorescent molecule with absorption and emission spectra in the visible to near-IR spectral range, or any molecule capable of generating a signal suitable for imaging purposes. In some embodiments, the imaging tag can include a radiolabel as well as a fluorescent label.

In general, an amino group of any embodiment of the N-heterocyclic bisphosphonates, or the phosphonocarboxylate analogues thereof, can be attached to an imaging tag such as a fluorescent dye by using a fluorescent dye that includes a carboxylic acid group that has been converted to an activated ester derivative susceptible of facile reaction with an amino group.

### BRIEF DESCRIPTION OF THE DRAWINGS

For a more complete understanding of the present invention, reference is now made to the following descriptions taken in conjunction with the accompanying drawings, in which:

FIGS. 1A-1B show a representation of Scheme 1, depicting the synthesis of a fluorescent bisphosphonate ‘toolkit’. In FIG. 1A, the conditions are A(1): ˜5% MeOH/H₂O, 40-50° C.; A(2): 1:1 TFA/H₂O, RT; B(1): H₂O, RT; B(2): NH₃.H₂O, RT; (C): FAM, SE (8), RhR-X, SE (9), ROX, SE (10), AF647, SE (11) or 800 CW, SE (12), NaHCO₃/DMF, pH 8.3-9.0, RT, in darkness. In FIG. 1B, particular embodiments are described.

FIG. 2 is a schematic drawing of a bifunctional azido-containing N-heterocyclic bisphosphonate (amino-azido-para-dRIS). The shaded star and triangle represent the same or different linked materials, such as a fluorescent tag, drug molecule of interest, peptide, protein, oligonucleotide, nanoparticle or polymer.

FIG. 3 is a graph showing the results of the HAP column binding assay of fluorescent BP probes. Data are shown as mean±SD from three individual studies, as relative retention times normalized to MS (FAM conjugates; 5 (6)-RhR conjugates; 5 (6)-ROX conjugates; AF647 conjugates; and 800 CW conjugates are shown).

FIG. 4 is a panel of graphs of adsorption isotherms for the binding of four fluorescent BP/PC probes on an HAP column. In the figure, FIG. 4A: 5 (6)-ROX-RIS; FIG. 4B: 5 (6)-ROX-RISPC; FIG. 4C: AF647-RIS; FIG. 4D: AF647-RISPC to HAP at pH 6.8, and include Scatchard plots of the same data below each respective adsorption isotherm graph; data are mean±SD (n=3).

FIG. 5 is a panel of results of prenylation assay and J774.2 cell viability assay of some fluorescent BP imaging probes. In the figure, FIGS. 5A-5C show results of Western blot assays for unprenylated Rap1A (uRap1A). J774.2 macrophages were treated with 10 or 100 μM of fluorescent analogues of MS (A), dRIS (B), and ZOL(C), the respective native BP, or vehicle, for 24 h. Detection of β-actin served as loading control. The ratio between abundance of unprenylated Rap1A and β-actin is indicated for each sample below the blots. FIGS. 5D-5F provide results of cell viability assays of J774.2 macrophages. Cells were then treated with 10, 100 or 500 μM of fluorescent analogues of RIS (D), dRIS (E), and ZOL (F), the respective native BP, or vehicle, for 48 h. Results are shown as mean±SD of ≧2 independent experiments, performed at least in duplicate.

### DETAILED DESCRIPTION

In some embodiments, a bone imaging toolkit, for example containing 21 fluorescent probes with variable spectroscopic properties, bone mineral binding affinities, and pharmacological activities, can be created, using two complementary linking strategies. The linking chemistry allows attachment of a wide selection of fluorescent dyes in the visible to near-infrared range to any of the three clinically important heterocyclic bisphosphonate bone drugs (risedronate, zoledronate and minodronate or their analogues). The resultant suite offers great flexibility and multiple options to “mix and match” fluorescence emission wavelength, relative bone affinity and presence or absence of anti-prenylation activity of the probes, used alone or in combination for bone-related imaging applications.

Recently, the inventors introduced a so-called ‘magic linker’ synthesis (Scheme 1, route A) [13], to prepare the first example of a fluorescently labeled heterocyclic N-BP, formed from RIS (1a) or related phosphonocarboxylate (PC, 1b) analogues and 5(6)-carboxyfluorescein (8). The synthesis crucially centered on using the epoxide derivative 5 to attach a universal linker group to the heterocyclic bisphosphonate drug under exceptionally mild conditions (pH near neutral, aqueous alcohol, 40° C.) with good regioselectivity. After deprotection of the products 2a-2c, the resulting drug-linker conjugates 4a-4c advantageously included a) a primary amine on the linker moiety for facile conjugation to the activated ester of an imaging dye, b) a positively charged pyridinium nitrogen to mimic the FPPS carbocation intermediate [14] in enzymatic catalysis, and c) an additional hydroxyl group to counteract decreased aqueous solubility associated with the addition of the hydrophobic alkyl chain [13].

It has proved desirable to greatly extend this approach to more diverse N-BPs and their analogues, coupled to fluorophores with a wide range of structural and spectroscopic properties, thus generating a fluorescent bisphosphonate ‘toolkit’ to enable biological experiments where different probes may be visualized within the same imaging assay. Some embodiments provide the straightforward and adaptable construction of a fluorescent bisphosphonate probe ‘toolkit’ that includes all three clinical heterocyclic N-BP drugs using complementary synthetic routes. The probes can be prepared in good yields (50-77%) and high purity (>95%), and can be fully characterized by ¹H, ³¹P NMR, UV-VIS, fluorescence emission, high resolution mass spectrometry and HPLC. Similar probes are being applied to studies of the mechanism of ONJ [15], otoscleorosis [16], and the discovery of the role of macrophages in bisphosphonate trafficking in tumor cells [17].

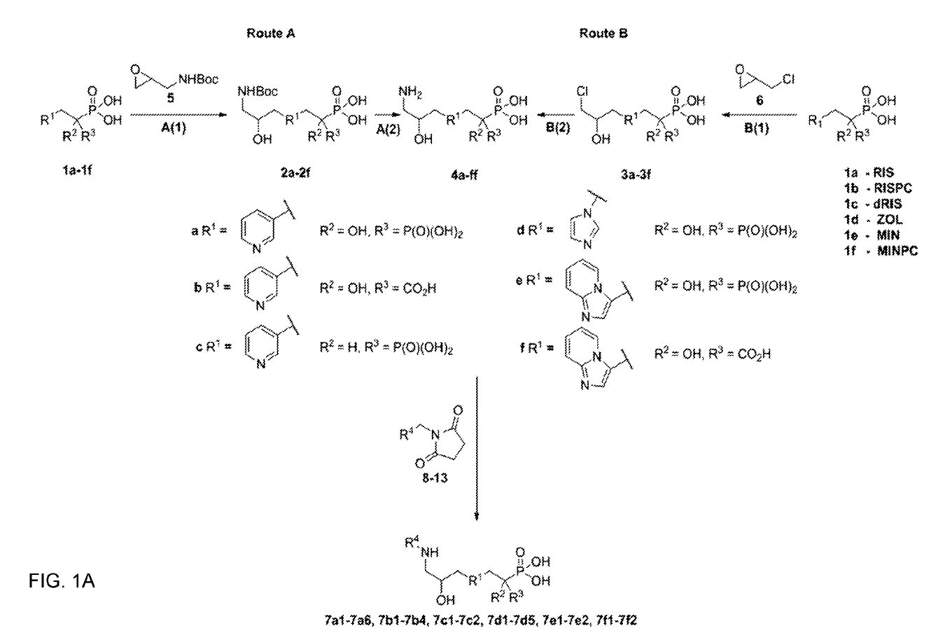

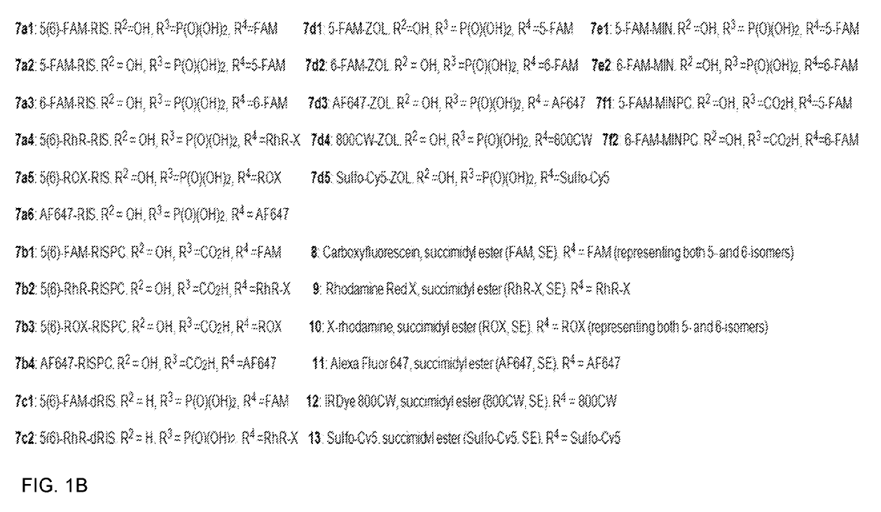

Alternate approaches to construct the fluorescent bisphosphonate “toolkit” are depicted in Scheme 1 of FIG. 1. The N-BPs and PCs (1a-1f) can be conjugated with the appropriate “magic linker” epoxide (5 or 6) via the original (A) or the new (B) route of synthesis, yielding the BP- or PC-linker intermediates, respectively, which can then be reacted with the activated ester of any of a diverse group of fluorescent dyes (for example, commercially available succinimidyl esters can be utilized), to afford the final fluorescent imaging probes.

In another aspect, dual functional bisphosphonates for use as imaging probes and other uses are provided, including the preparation of bifunctional amino/azido-containing N-heterocyclic bisphosphonate based on para-dRIS, which is adaptable to other N-heterocyclic deoxy-bisphosphonates and related analogues, such as dRIS, dRISPC, and the like. Two functionalities, azido and amino group, are introduced together which makes it possible for dual-conjugation, and the introduced alkyl chain only has three carbons in some embodiments, which could minimize the potential effect on HAP binding affinity caused by the intrinsic hydrophobic property of alkyl chains. The bifunctional hydroxy/azido-containing N-hetero bisphosphonate extends a general method to actual drugs such as, but not limited to, RIS, ZOL and MIN. Also, a quick and selective method to connect bisphosphonates above via a novel linker to many other molecules including fluorescent tags, drug cargos, peptides, proteins, oligonucleotides, nanoparticles, polymers, and the like, is provided. This provides a basis for sensitive detection of the drugs in bone/bone samples.

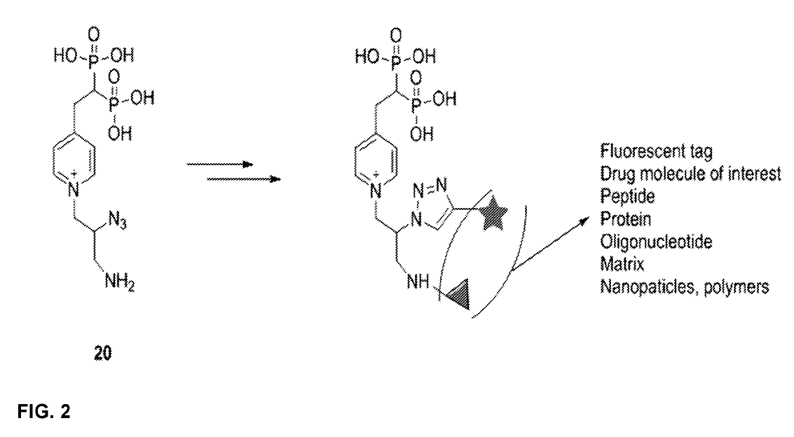

In some embodiments, the synthesis of a bifunctional amino/azido-containing N-heterocyclic bisphosphonate (amino-azido-para-dRIS, 20, and FIG. 2) is provided, with the compound having clickable reactivity for the preparation of fluorescent probes. The N-heterocyclic BP in these embodiments is an analogue of deoxy-RIS (para-dRIS), and two functionalities, amino and azido groups, are introduced sequentially, which makes it possible for dual-conjugation, e.g., reacting one group with an imaging tag and the other one with a drug, peptide, protein, oligonucleotide, fluorescent quencher, et al. In addition, the introduced alkyl chain in these embodiments only has three carbons, in order to minimize the potential effect to HAP binding affinity caused by the intrinsic hydrophobic property of alkyl chains.

In a further aspect, azido-containing N-heterocyclic bisphosphonates containing a linker for detection of heterocyclic nitrogen-containing bisphosphonate are provided. Because there is no sensitive and convenient non-radioactive tracer analysis method currently available for detection of clinically used nitrogen-containing heterocyclic bisphosphonates, such as 1a-d, risedronate, zoledronate or minodronate, in bone, a method is developed that involves selectively linking an azido epoxide 31 with a nitrogen-containing heterocyclic bisphosphonate to form the linker intermediate 32, as represented in Scheme 2 depicting the synthesis and click reaction of azido-RIS and a tagged-alkyne. Compound 32 can then be connected to a fluorescent dye or other label containing a terminal alkyne group (33) to form the linked conjugate 34 or, alternatively, the azido group can be reduced to a primary amino group by known methods such as, but not limited to, catalytic hydrogenation. The formation of the primary amino group will allow for conjugation to add a fluorescent or other label containing an activated group, such as a succinimidyl ester. The resulting nitrogen-containing bisphosphonate fluorophore conjugate can be detected by its emission fluorescence. In the example in Scheme 2, N-heterocyclic bisphosphonate 1a (risedronate) is coupled to glycidyl azide 31 to give azido-containing bisphosphonate-linker 32, which can then be reacted with fluorescent labeled alkyne 33 to afford a fluorescently labeled bisphosphonate 34.

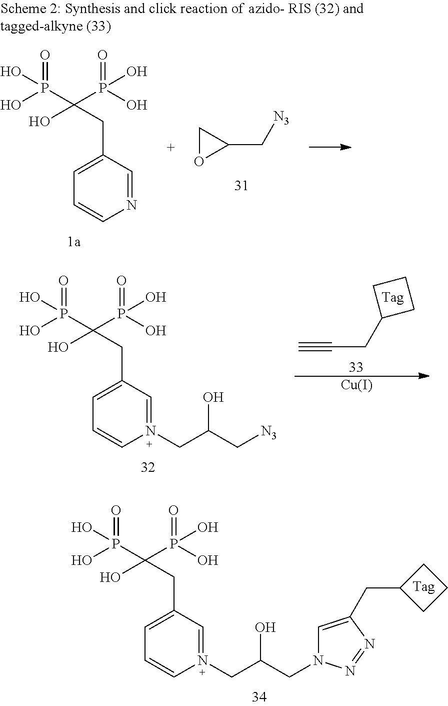

In various aspects, a fluorescent bone-imaging probe ‘toolkit’ is synthesized. A linking strategy with a new route is developed, which together with the original “magic linker” method, makes possible attachment of activated fluorescent dyes to all of the clinically relevant heterocyclic N-BPs and related analogues, under mild conditions. All the fluorescent probes can be prepared in good yield (50-77%) and high purity (>95%). They generally retain substantial affinity for bone mineral, reflecting the varying affinities of their parent drugs. The conjugated fluorophores exert some influence (generally a slight reduction, but in one case enhancement) on the mineral affinity of the probes, which is a consideration when interpreting data generated with such probes. Conjugates with FAM substantially retained the anti-prenylation activity of the parent BP, making it possible to correlate biological activity with localization using these probes. The diverse pharmacological and spectroscopic properties of the probes comprising the “toolkit” make them highly useful, for example, in bone-related imaging studies [15a,17,25,27-28].

In some embodiments, a bifunctional amino/azido-containing N-heterocyclic bisphosphonate (amino-azido-para-dRIS, 7) has been synthesized; and has been successfully applied in the preparation of fluorescent probes via the CuAAC click reaction. The synthetic strategy based on para-dRIS, can be extended to other N-heterocyclic deoxy-bisphosphonates and related analogues, such as dRIS, dRISPC, and the like. In addition, two functionalities, azido and amino group, can be introduced together via the strategy, which makes it possible for dual-conjugation, and the introduced alkyl chain can only have three carbons in some embodiments, which could minimize the potential effect on HAP binding affinity caused by the intrinsic hydrophobic property of alkyl chains.

In embodiments related to treatment, embodiments of the compounds of the present invention may be formulated as pharmaceutical compositions. Pharmaceutical compositions comprise a compound and a pharmaceutically acceptable carrier and/or diluent. The compound is present in the composition in an amount which is effective to treat a particular disorder of interest, and preferably with acceptable toxicity to the patient. Typically, the pharmaceutical composition may include a compound of this invention in an amount depending upon the route of administration. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. For compositions formulated as pills, capsules, granules, or tablets, the composition can contain, in addition to the active compound, other substances such as dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

The present invention may be better understood by referring to the accompanying examples, which are intended for illustration purposes only and should not in any sense be construed as limiting the scope of the invention.

### Example 1

Scheme 1 in FIG. 1 shows alternate approaches for the construction of a fluorescent bisphosphonate “toolkit”.

The nucleophilicity and Lewis basicity of the nitrogen atom of the three heterocyclic N-BPs studied in this report vary [18], and these properties appear to be the major determinants of their conjugation reactivity. Thus, the ‘magic linker’ methodology for RIS required modification for ZOL (1d), MIN (1e), and their related analogues (1f). The rank order of ΔG₀^{≠} (defined as the activation free energy of processes with ΔG⁰=0) is as imidazoles>pyridines>1-azabicyclooctanes, indicating that the reorganization energies for the reactions of imidazoles with electrophiles are significantly higher than those for the other amines and that imidazoles are less nucleophilic than pyridines of comparable basicity [19]. The protonation status of the heteroatom at different pH is also critical to the reactivity. For example, the pKa of the heterocyclic nitrogen of RIS, ZOL and MIN is 5.67, 6.67, and 6.54, respectively [20]; in the previous ‘magic linker’ synthesis of RIS, the pH of reaction mixture for the linking step was adjusted to 6.0. Under such conditions, more than 50% (˜71.3% calculated) of the nitrogen of RIS will be deprotonated, and able to attack the epoxide ring nucleophilically [13]. However, at pH 6.0, only 18% of the nitrogen of ZOL (22% for MIN) is deprotonated, thus the reactions were very slow (2d) at pH 6.0. At pH 7.0-7.5, reaction was more rapid, but the regioselectivity (N-alkylation vs. O-alkylation) for 1d, 1e, 1f was not as good as for RIS and its analogues (1a, 1b, 1c); 10-20% of side products could be observed, which were confirmed by HPLC, NMR and MS to be the products of N,O(P)-dialkylation (O-alkylation occurs between the phosphonate/carboxylate group of the drug and the epoxide). The reaction rate of MinPC (1f)-epoxide (5) coupling was even slower than ZOL under the same reaction conditions (same reactant ratio, pH 7.5, 40-45° C.), which might be due to a steric effect from the large heterocycle. In addition, the percent of side products was higher and O-alkylation products were also observed in the reaction mixture.

Commercially available epichlorohydrin 6 was therefore explored as a new linker precursor. Besides being inexpensive, it is more water-soluble and more reactive compared to epoxide 5. Epichlorohydrin was reacted with compounds 1a-1f, yielding intermediates 3a-3f, which were ammonolyzed to produce the final drug-linker conjugates 4a-4f. The conjugation (Scheme 1, Route B, 1a-1f→3a-3f) proceeded with a higher rate compared to the previous route (Scheme 1, Route A, 1a-1f→2a-2f) under the same reactant ratio and pH, even at rt. The percentage of side-products for 1d-1f was similar to that in Route A, Formation of side-products was not observed in the reaction of 1a with 5 equiv. of epichlorohydrin (FIG. S5). Therefore, it was decided to retain Route A for synthesis of 4a-4c, while preferring Route B for synthesis of 4d-4f. The reactant ratio of 1:5 (drug:linker (6)) at appropriate pH (7.4-8.0, depending on the drug) was adopted. In at most 20 h at rt, 70-85% of 4d-4f was obtained. Pure 4d-4f for conjugation with activated fluorescent dyes in the following step were obtained by preparative anion exchange HPLC.

Demberelnyamba et al. reported [21] that reaction of pyridine or imidazole with epichlorohydrin in acetonitrile resulted in nucleophilic attack of the pyridine or imidazole nitrogen to displace chlorine to form an epoxide derivative, instead of opening the epoxide ring, whereas we find that reaction of N-BPs with epichlorohydrin under aqueous conditions yields solely the epoxide-opened alkyl chloride product.

The inventors were able to reproduce the result of Demberelnyamba et al., reacting pyridine with epichlorohydrin in acetonitrile to obtain exclusively product B (Scheme 3), identified by mass-spectrometry and ¹H NMR (however, the ¹H NMR for B obtained by the inventors was different from that obtained by Demberelnyamba et al.).

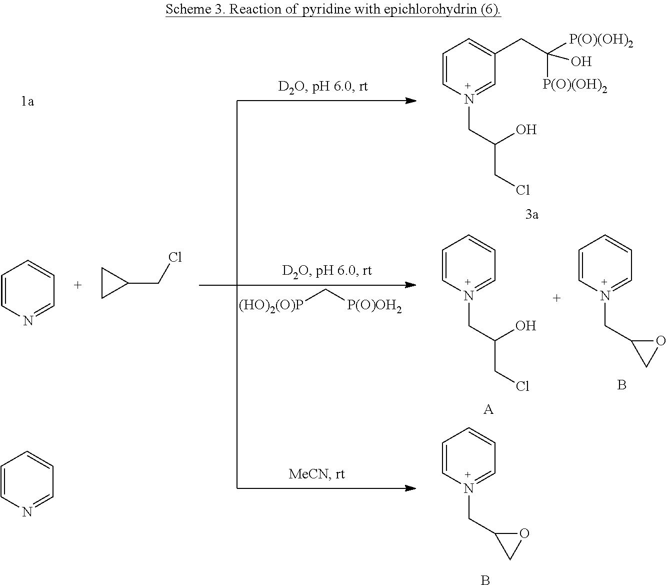

Surprisingly, when pyridine was reacted with epichlorohydrin in water in a 1:1 ratio to mimic the conditions used for risedronate—epichlorohydrin conjugation, adjusting the pH to 6 with methylenebisphosphonic acid, the inventors obtained a mixture of conjugates A and B (Scheme 3), in a ratio of 6:1, and at reaction ratio of 1:5 (pyridine:epichlorohydrin), the ratio fell to A:B=1:2, suggesting that the BP moiety strongly influences the regioselectivity observed with RIS.

A series of fluorescent dyes with distinguishable emission spectra, including three commercially available near infrared dyes, Alexa Fluor® 647 (AF647), Sulfo-Cy5 and IRDye®800CW (800CW), was selected to generate the fluorescent bisphosphonate probe ‘toolkit’. Conjugation was carried out between the drug-linker intermediates (4a-4f) and the succinimidyl ester (SE) of the fluorescent dyes under similar reaction conditions (Scheme 1, step iii) for the different fluorescent dyes, with minor modifications to reactant ratio and reaction pH. The reactions proceed quickly and can be monitored by TLC conveniently (100% MeOH as eluent). To obtain pure fluorescent bisphosphonate probes (7a-7f), chromatographic isolation was generally effective. Except for the carboxyfluorescein (FAM, 8) and Rhodamine X-Red (RhR-X, 9) conjugates, which required preparative TLC (100% MeOH as eluent) to remove free dye label from the product mixture, all the fluorescent conjugates could be directly purified by preparative reverse phase HPLC in one pass (supplemental data). All the final fluorescent conjugates were fully characterized by HPLC, UV-VIS and fluorescence emission spectroscopy, ¹H and ³¹P NMR and high-resolution MS. It is noteworthy that the isomeric 5- and 6-carboxyfluorescein (FAM, 8) conjugates can be directly synthesized from their respective pure FAM, SE isomers, or alternatively and less expensively separated from the mixed isomers of the FAM conjugates.

The prepared probes fluoresce at widely different optical wavelengths (Table 1), allowing for simultaneous detection of individual low and high bone affinity BPs and PCs in cells and tissues. The pharmacologically relevant properties, in particular the HAP affinity of the probes and their effects on protein prenylation, were investigated to guide probe selection for different applications.

TABLE 1
Spectroscopic properties of fluorescent bisphosphonate probes

| | Maximum absorption | Maximum emission | |
|---|---|---|---|
| | wavelength (λmax | wavelength (λmax | Extinction coefficient |
| Probes | (abs), nm)^{[a]} | (em), nm)^{[a]} | (M⁻¹cm⁻¹)^{[b],[c]} |
| 5(6)-FAM-RIS (7a1) | 493 | 518 | 73,000 (pH 7.2) |
| 5-FAM-RIS (7a2) | 493 | 521 | 73,000 (pH 7.2) |
| 6-FAM-RIS (7a3) | 493 | 517 | 73,000 (pH 7.2) |
| 5(6)-RhR-RIS (7a4) | 567.5 | 589 | 114,850 (pH 7.5) |
| 5(6)-ROX-RIS (7a5) | 580 | 606 | 72,000 (pH 8.0) |
| AF647-RIS (7a6) | 648 | 666 | 240,000 (pH 7.0) |
| 5(6)-FAM-RISPC (7b1) | 493 | 518 | 73,000 (pH 7.2) |
| 5(6)-RhR-RISPC (7b2) | 568 | 589 | 114,850 (pH 7.5) |
| 5(6)-ROX-RISPC (7b3) | 579 | 606 | 72,000 (pH 8.0) |
| AF647-RISPC (7b4) | 648 | 666 | 240,000 (pH 7.0) |
| 5(6)-FAM-dRIS (7c1) | 493 | 518 | 73,000 (pH 7.2) |
| 5(6)-RhR-dRIS (7c2) | 567.5 | 589 | 114,850 (pH 7.5) |
| 5-FAM-ZOL (7d1) | 493 | 521 | 73,000 (pH 7.2) |
| 6-FAM-ZOL (7d2) | 493 | 516 | 73,000 (pH 7.2) |
| AF647-ZOL (7d3) | 648.5 | 666 | 240,000 (pH 7.0) |
| 800CW-ZOL (7d4) | 774 | 789 | 240,000 (pH 7.0) |
| Sulfo-Cy5-ZOL (7d5) | 644 | 663 | 271,000 (1x PBS, pH 7.4) |
| 5-FAM-MIN (7e1) | 493 | 522 | 73,000 (pH 7.2) |
| 6-FAM-MIN (7e2) | 493 | 518 | 73,000 (pH 7.2) |
| 5-FAM-MINPC (7f1) | 493 | 522 | 73,000 (pH 7.2) |
| 6-FAM-MINPC (7f2) | 493 | 517 | 73,000 (pH 7.2) |

^{[a]}There is ± 1 nm error of λmax (abs) and λmax (em).
^{[b]}The extinction coefficient of each probe is assumed the same as its corresponding fluorescent dye.
^{[c]}Unless specified, 0.1M phosphatebuffer is used for all the measurements

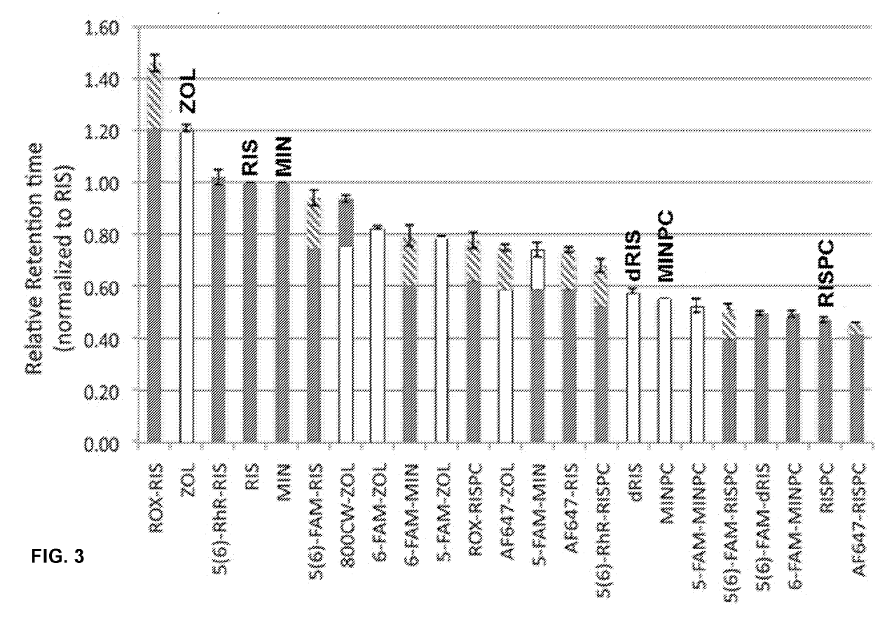

The mineral binding affinity of BPs is predominantly determined by the phosphonate groups, while an R¹—OH group may further enhance the affinity [1a,22]. This is demonstrated by a binding affinity comparison of RIS, RISPC (in which one phosphonate was replaced by a carboxylate) and dRIS (in which R¹ is H), where the rank order of affinity is RIS>dRIS>RISPC [23]. To investigate whether the attached fluorophore influences the mineral binding affinity, the retention time of each fluorescent BP conjugate on a HAP column was measured and normalized to the retention time of RIS. As shown in FIG. 3, the fluorescent conjugates have similar binding affinity to the parent compounds, although 5(6)-ROX conjugates display slightly higher affinity than their counterparts. AF647-RIS exhibits the largest relative decrease of HAP affinity compared to other fluorescent RIS conjugates, but still retains strong absolute binding affinity.

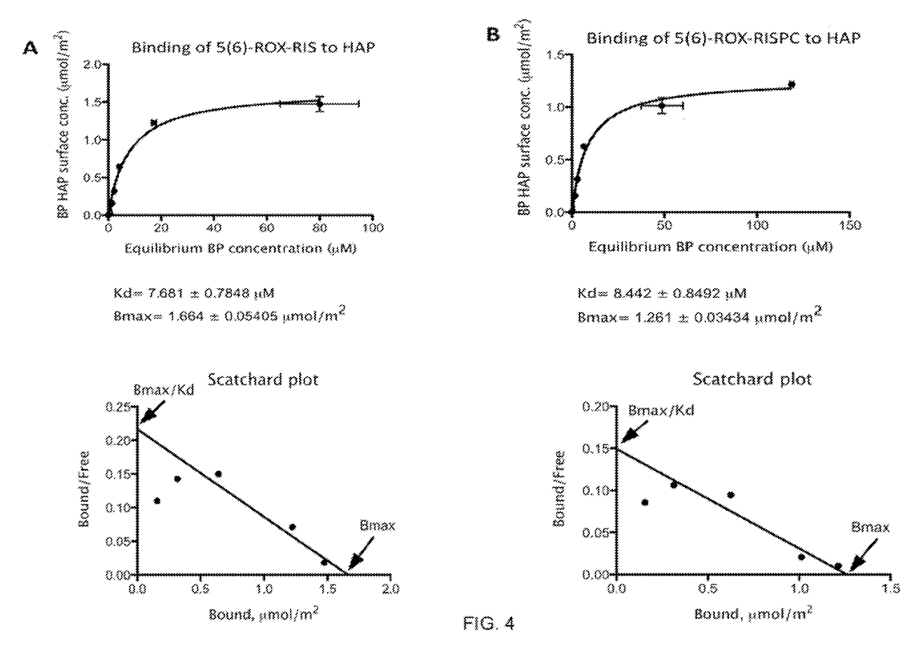

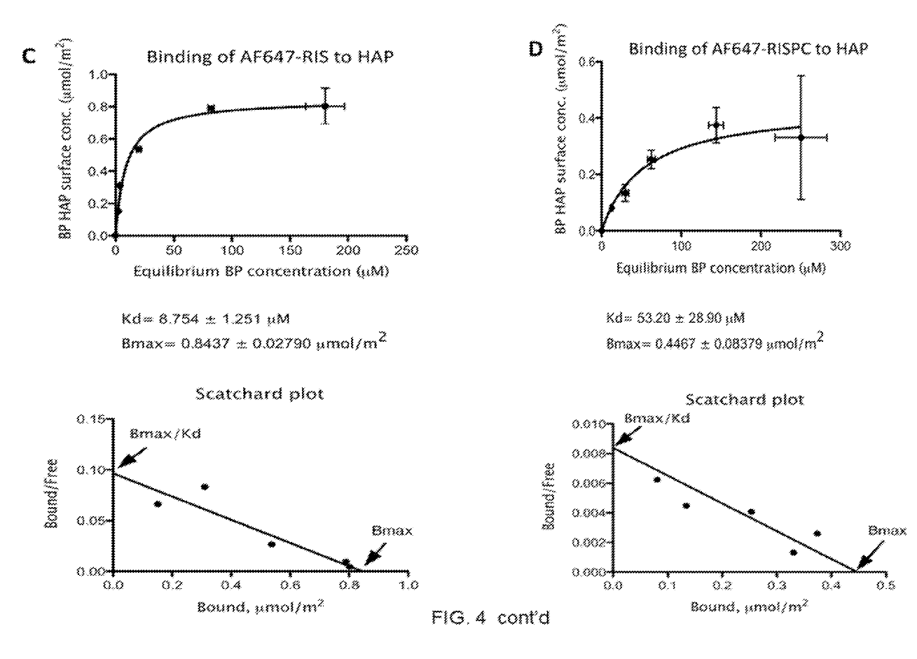

The affinity rank order of RIS, dRIS and RISPC conjugates with the same fluorophore remains the same as RIS>dRIS/RISPC, although the difference between dRIS and RISPC conjugates was not statistically significant. In addition, measurement of dissociation constants (K_{d}) and maximum capacities of 5(6)-ROX-RIS, 5(6)-ROXRISPC, AF647-RIS and AF647-RISPC for HAP by Langmuir adsorption isotherms [24] are in good accordance with the results from the HAP column assays (FIG. 4) and from earlier in vitro dentine-binding assays [25].

The results for ZOL and its fluorescent conjugates are similar to those for RIS (FIG. 3). 6-FAM-ZOL had a slightly longer retention time on the HAP column than its 5-isomer, in accord with the results for 6-FAM-RIS and 5-FAM-RIS [13], while 6-FAM-MIN and 5-FAM-MIN (FIG. 3) were similar in affinity.

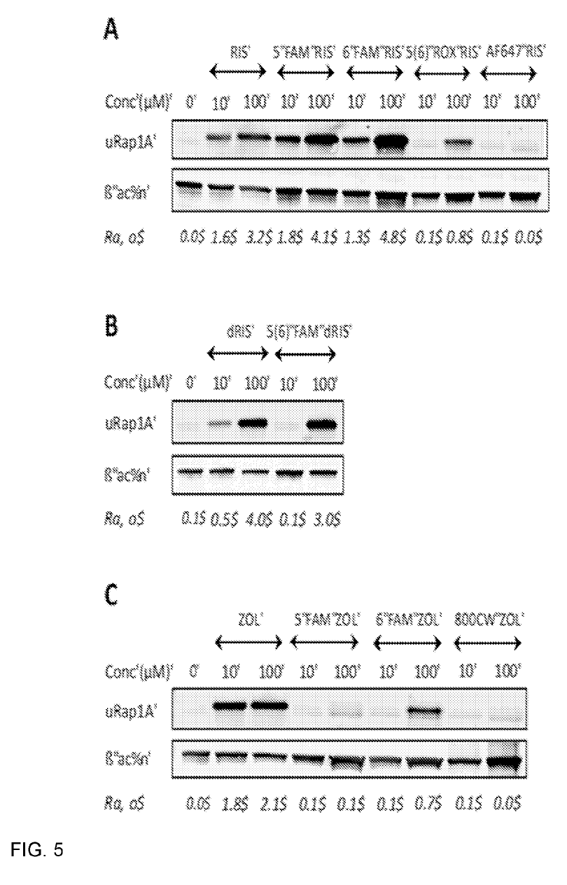

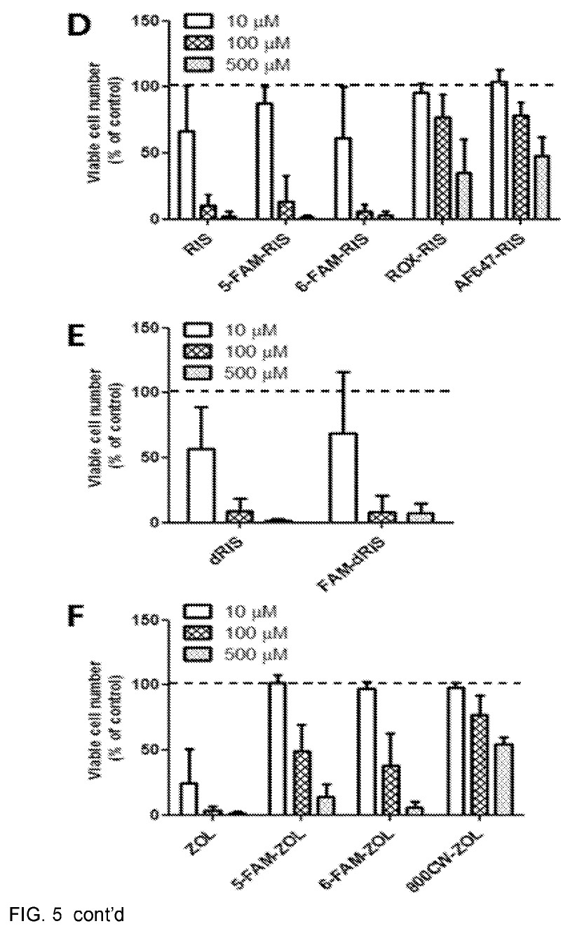

A key pharmacological activity of N-BP drugs is their inhibition of protein prenylation and indirectly osteoclast-mediated bone resorption. The detection of unprenylated Rap1A (uRap1A) and cell viability of J774.2 macrophages have been used widely as in vitro screening assays to assess the pharmacological activity of novel BP and related analogues [13,26]. PC analogues (RISPC, MINPC) are much less active than the N-BPs in these assays; thus only fluorescent BP and deoxy-BP (dRIS) conjugates were analyzed (FIG. 5).

Unprenylated RaplA was clearly detectable in cell lysates from both 5-FAM-RIS- and 6-FAM-RIS-treated J774.2 mouse macrophages (FIG. 5A), suggesting they retain the ability to affect the mevalonate pathway as previously reported in cultured J774.2 macrophages [13] and in osteoclasts isolated from 5(6)-FAM-RIS-treated rabbits [27]. 5(6)-FAM-dRIS was also active, with potency comparable to its parent BP dRIS (FIG. 5B). Both 5-FAM-ZOL and 6-FAM-ZOL retain some activity, although their potencies are weaker than native ZOL (FIG. 5C). Anti-resorptive activity of 5-FAM-ZOL was recently demonstrated in a rat model in vivo [15a]. 5(6)-ROX-RIS also shows activity (FIG. 5A). Cells treated with two near-infrared BP conjugates, AF647-RIS and 800CW-ZOL, did not show accumulation of unprenylated Rap1A at the concentrations used, suggesting these fluorescent BP probes are inactive. The results of the cell viability assay are in accordance with the prenylation assay (FIG. 5D-F), with the exception that AF647-RIS and 800CW-ZOL-treated J774.2 macrophages exhibit modest decreases in viable cell number at high concentrations, which may be due to a non-specific effect, e.g. calcium chelation lowering the availability of free ionic calcium in the growth medium. A steric effect of the large 800CW and AF647 fluorophores may explain the biological inactivity of these fluorescent BP conjugates.

### Example 2

### Bifunctional N-Heterocycle Bisphosphonates

The concept of “click chemistry” has gained lots of attention after it was first proposed by Sharpless, et al. [29], who also identified a number of reactions that meet the criteria for click chemistry, namely, reactions that “are modular, wide in scope, high yielding, create only inoffensive by-products (that can be removed without chromatography), are stereospecific, simple to perform and that require benign or easily removed solvent.” Of all these click reactions, the Huisgen 1,3-dipolar cycloaddition reaction of alkynes and azides to yield 1,2,3-triazoles is arguably “the cream of the crop” and stands at the “central stage” [30,31], especially when Cu(I) catalysis was found to dramatically accelerate the reaction as well as offer high regioselectivity [32,33]. The Cu(I) catalyzed alkyne-azide coupling (CuAAC) reaction has become undoubtedly the most powerful click chemistry reaction and has been applied widely in drug discovery, polymer and materials science, as well as bioconjugation [34-39].

It was known that the nitrogen atom in the R² side chain of clinically used bisphosphonates plays a pivotal role in their anti-resorptive pharmacological activity as discussed previously. CuAAC click reaction is a very efficient way to introduce 1,2,3-triazole heterocycle into a molecule, suggesting its potential for development of novel bisphosphonates. In addition, BPs can be used as “magic bullets” in drug delivery and imaging probe studies, and CuAAC reactions of alkynyl bisphosphonates or azido bisphosphonates should have applications in these studies, because the CuAAC reaction conditions are usually compatible with biological systems [39].

However, interest in preparing alkynyl/azido-containing bisphosphonates, such as compounds of the following formulas 14-18, only started in the last few years. It was not until 2007 that Osipov and Roschenthaler [40] reported the first application of CuAAC click chemistry in bisphosphonate synthesis using tetraethyl but-3-yne-1,1-diyldiphosphonate (14) or tetraethyl hepta-1,6-diyne-4,4-diyldiphosphonate (15), which is five years after the introduction of CuAAC reaction; although a series of azidoalkylphosphonates, -phosphinates and -phosphine oxides were synthesized and their 1,3-dipolar cycloaddition reactions were investigated earlier [41]. Wiemer et al. recently reported the use of the CuAAC reaction for syntheses of triazole-based inhibitors of geranylgeranyltransferase II from the same alkynyl bisphosphonate starting material 14 [42]. Guenin et al. synthesized HMBPyne (16) and applied the compound in the coating of an iron oxide nanoparticle γ-Fe₂O₃ to act as an anchored scaffold ready for further “click” modification [43]. McKenna et al. reported the first examples of α-azido bisphosphonate esters and acids (17a-d) and applied the α-azido bisphosphonic acid in the preparation of novel nucleotide analogues containing a CHN₃ or C(CH₃)N₃ at either the α,β or β,γ bridging position; but their CuAAC reactions have not been reported [44]. Herczegh et al. utilized the O-Silylated 3-azidopropyl-tetraethyl bisphosphonate (18) and synthesized a series of 1,2,3-triazolelinked hydrobisphosphonate derivatives of ciprofloxacin as antibacterial agents [45]. Chen et al. recently reported the synthesis of β-azido bisphosphonate (5) in EtOH-water (1:1) (19), and investigated one-pot synthesis of triazole bisphosphonates which were proposed via the intermediate compound 18 [46]. It should be noted that Szajnman and Rodriguez et al. reported that compound 18 was not afforded in solvents such as methanol, methanol-water (1:1) or acetonitrile, from the same starting materials as Chen et al. used [47].

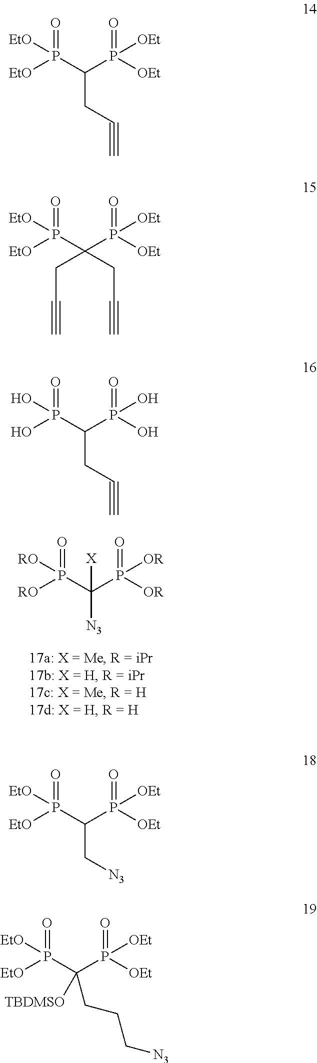

Another way to introduce an alkynyl or azido group into some bisphosphonates, which could be further conjugated with target molecules of interest by the CuAAC reaction, is by direct coupling of the alkynyl/azido containing reagents with the terminal amino group of bisphosphonates such as alendronate and pamidronate [48-51]. However, to the best of the inventors' knowledge, there is no report on alkynyl/azido-containing N-heterocyclic bisphosphonates yet in literature.

The synthesis of amino-azido-para-dRIS is outlined in Scheme 4. The readily available tetraisopropyl methylene bisphosphonate (21) was treated with NaH to generate carbanion and was then reacted with 4-(chloromethyl)pyridine (22), yielding para-dRIS (23). The reaction is rather slow at room temperature (r.t.) probably due to steric hindrance, thus temperature is increased to 70° C. to accelerate the reaction and improve yield. In addition, besides mono-substituted compound 23, a little di-substituted compound was also formed. Compound 23 was first purified by column chromatography and partially dealkylated product was observed, suggesting the phosphonate ester is hydrolyzed on silica gel column. Since the ester protection is necessary for the following reactions, an extraction/wash procedure was developed to avoid the column chromatography.

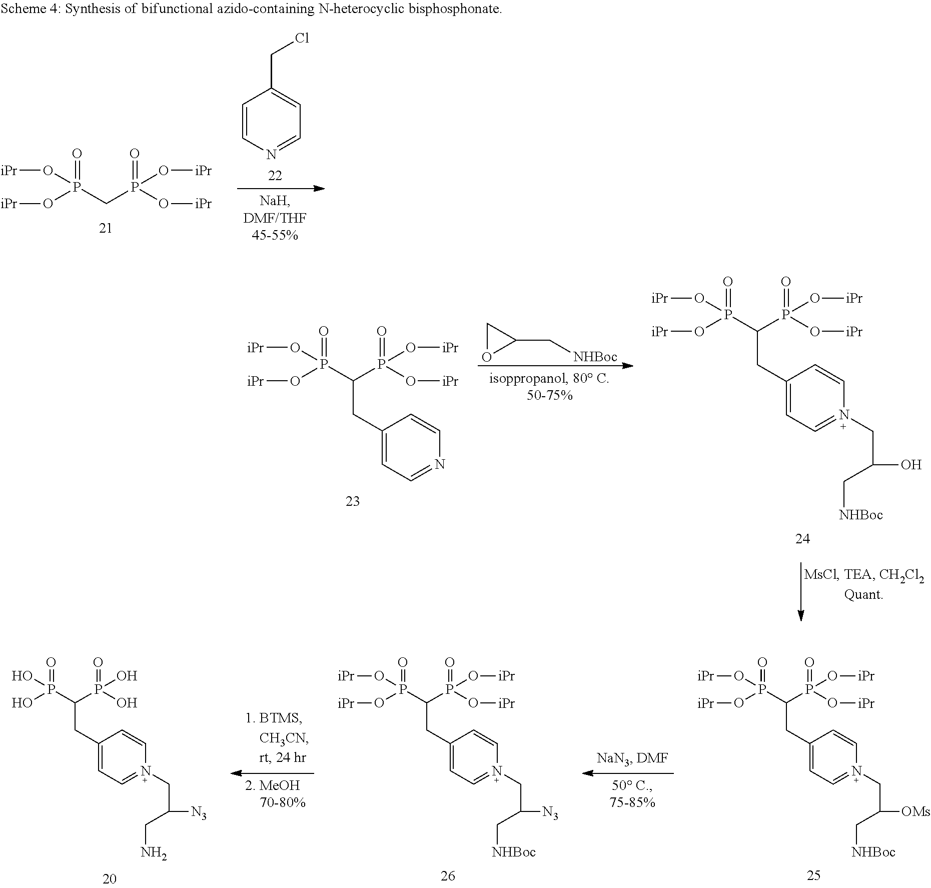

Compound 24 obtained after extraction purification was used for preparation of NHBoc-azido-para-dRIS (26) via the intermediate compound 25. It was found that at temperature >60° C., ˜21% of compound para-dRIS (10) was observed in the reaction mixture, suggesting the C—N bond cleavage. Notably, compound 24 is fairly stable and no C—N bond cleavage was seen, implying the azido group in proximity has a potentially catalytic role in the C—N bond cleavage. When the temperature decreased to 50° C., C—N bond cleavage was minimized, indicating the stability of compound 26 is temperature dependent. Finally, the isopropyl groups were deprotected by BTMS method after optimization, and the products were further purified by HPLC, giving the target molecule amino-azido-para-dRIS (20). It should be noted that this synthetic route is also applicable to other N-heterocyclic deoxy-bisphosphonates and related analogues, such as dRIS, dRISPC, etc.

The clickability of amino-azido-para-dRIS (20) was investigated by reacting with an alkyne-containing fluorescent dye (5(6)-FAM-alkyne (29), synthesized according to Scheme 5). Reactions in H₂O at different temperatures (r.t. and 55° C.) were tried first and <10% of triazole product (30) was found after 48 hrs. Obvious precipitates could be observed before and after the reaction, which were proposed as complexes with Cu(II) from CuSO₄ that has not been converted to Cu(I) completely yet or oxidized later by tiny amount of O₂; thus the reaction was tried by adding Cu(I) catalyst that was already prepared ahead of time and running the reaction under vacuum line to avoid the introduced O₂. However, precipitates still existed after all reactants were mixed. It was then found that the precipitates were due to poor solubility of 5(6)-FAM-alkyne (29) and triazole product (30) in aqueous solutions if pH was lower than 8. Thus 0.2 M triethylammonium bicarbonate buffer (pH 8.0) was used as reaction medium (Scheme 5) and no precipitates were observed as the reaction went on. TLC analysis (100% MeOH as eluent) of the reaction mixture suggested that almost all 5(6)-FAM-alkyne were consumed and converted to triazole product (30) after overnight incubation at either r.t. or 45° C.

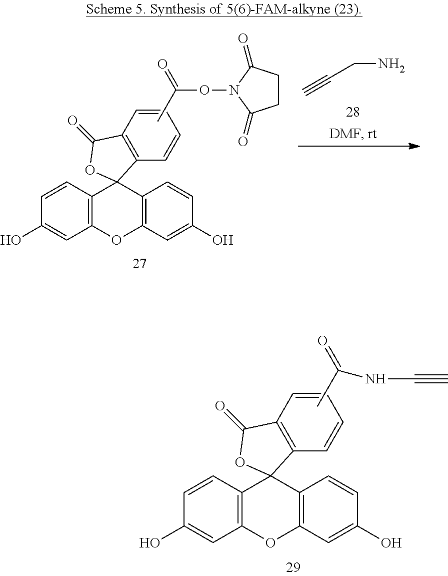

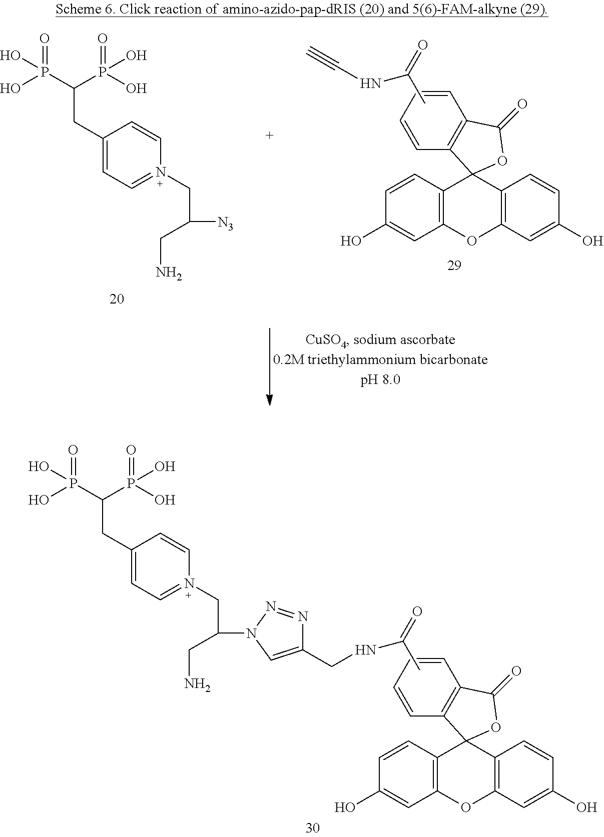

Since pH also influences the solubility of triazole product (30), a precipitation procedure by adjusting pH is used to purify the compound. The pH of reaction mixture was adjusted to 3.0 by 0.5 M HCl until no more precipitate formed. Precipitates were then collected by centrifuging and then washed sequentially by acetone (0.5 mL×2) and diluted HCl (pH=3.0, 0.25 mL×2). 5- and 6-isomers of product 30 were further separated by semi-preparative reverse phase HPLC.

### Example 3

Reagents and Spectral Measurements:

5(6)-, 5-, and 6-carboxyfluorescein, succinimidyl ester (FAM, SE) were purchased from Sigma Aldrich or Invitrogen, US. 5(6)-Rhodamine Red-X, SE (5(6)-RhR, SE), 5(6)-carboxy-X-Rhodamine, SE (5(6)-ROX, SE) and Alexa Fluor 647, SE (AF647, SE) were purchased from Invitrogen, US; sulfo-Cy5, SE was purchased from Lumiprobe, US, and IRDye® 800CW, SE was purchased from LI-COR Biosciences, US. Compounds 1a-1c were kind gifts from Warner Chilcott Pharmaceuticals (former P&G Pharmaceuticals). Compound 1d (zoledronic acid) was purchased from Molekula, UK. Compound 1e (minodronic acid) was purchased from Shanghai Hengrui International Trading Co. LTD, PRC. Compound 1f (3-IPEHPC) was synthesized in our lab according to a published procedure [52]. All other compounds were purchased from Aldrich or Alfa Aesar. Triethylamine (TEA) was distilled from KOH; CH₂Cl₂ was distilled from P₂O₅; and allylamine was distilled under N₂. All other compounds were used as supplied by the manufacturer. Thin layer chromatography was performed on Merck Silica Gel 60 F₂₅₄ plates, and the developed plates were visualized under a UV lamp at 354 nm. HPLC separations were performed on a Rainan Dynamax Model SD-200 system with a Rainan Dynamax absorbance detector Model UV-DII. NMR spectra were recorded on either 400 MHz Varian, 500 MHz Varian, 600 MHz Varian or 500 MHz Bruker spectrometers. UV spectra were recorded on a DU 800 spectrometer, and fluorescence emission spectra were taken on either a Jobin Yvon Horiba FluoroMax-3 fluorimeter equipped with a DataMax Software version 2.20 (Jobin Yvon Inc), Jobin Yvon Nanolog fluorimeter (Jobin Yvon Inc), SHIMADZU spectrofluorophotometer RF-5301PC, or PTI QuantaMaster model C-60SE Spectrometer equipped with a 928 PMT detector. High resolution mass spectra were performed by Dr. Ron New at UC Riverside High Resolution Mass Spectrometry Facility on a PE Biosystems DE-STR MALDI TOF spectrometer with a WinNT (2000) Data System. Other mass spectra were taken on ESI Thermo-Finnigan LCQ DECA XPmax Ion Trap LC/MS/MS spectrometer. See other references [53-54].

### Synthesis of Drug-Linker Intermediates 4a-4c

General Procedure:

The parent drug (1a-1c) was dissolved in water and the pH adjusted to 5.7-6.0 with 1 M NaOH. Epoxide 5 was dissolved in minimal methanol (MeOH) and added to the water solution, causing a slight precipitation to occur. The precipitate disappeared on heating (40-50° C.) and as the reaction progressed. The reaction was monitored by ³¹P NMR. After 90-95% of the desired product was obtained (³¹P NMR), the solvent was removed in vacuo, and the resulting white powder washed with diethyl ether, filtered, and dried in a dessicator. Standard deprotection was performed with 1:1 trifluoroacetic acid (TFA): H₂O. After the reaction mixture was stirred for 3-4 h at RT, the solvent was removed in vacuo, and the resulting crystals washed with diethyl ether and MeOH to yield the drug-linker intermediates.

### Synthesis of 1-(3-amino-2-hydroxypropyl)-3-(2-hydroxy-2,2-diphosphonoethyl)pyridinium (4a)

The monosodium salt of (1-hydroxy-2-pyridin-3-ylethane-1,1-diyl)bis(phosphonic acid), 1a (288 mg, 0.94 mmol, 1.00 eq), was dissolved in 4 mL water, and the pH adjusted to 6.2 with 1 M NaOH. To this solution, 164 mg of 5 (0.94 mmol, 1.00 eq) in minimal MeOH was added. The reaction mixture was stirred at 40° C. for 18.5 h, yielding 90% of 2a by ³¹P NMR. The solvent was removed in vacuo, and the residue was washed with diethyl ether, filtered, and dried in a desiccator. 2a, a white solid, was then used without further purification. ¹H NMR (D₂O): δ 8.68 (s, 1H), 8.46 (d, J=6.3 Hz, 1H), 8.42 (d, J=8.1 Hz, 1H), 7.78 (dd, J=8.2, 5.8 Hz, 1H), 4.67-4.62 (part. obscured by HDO, about 1H), 4.27 (dd, J=13.6, 9.6 Hz, 1H), 4.13-3.92 (m, 1H), 3.41-3.10 (m, 4H), 1.31 (s, 9H). ³¹P NMR (D₂O) δ 16.55 (d, J=21.7 s Hz, 1P), 16.33 (d, J=21.9 Hz, 1P).

The entire sample of 2a was dissolved in 50:50 water:TFA (v/v). After the solution was stirred at RT for 3 h, a 100% yield of 4a was achieved according to ¹H NMR. The solvent was then removed in vacuo, and the resulting solids were washed with ether, filtered, and dried, yielding 4a as white crystals, which were used without further purification. ¹H NMR (D₂O): δ 8.71 (s, 1H), 8.54 (d, J=6.0 Hz, 1H), 8.44 (d, J=8.1 Hz, 1H), 7.84 (dd, J=8.1, 6.0 Hz, 1H), 4.74 (part. obscured by HDO, about 1H), 4.41-4.21 (m, 2H), 3.39-3.21 (m, 3H), 2.96 (dd, J=13.0, 9.9 Hz, 1H). ³¹P NMR (D₂O): S 16.35 (d, J=26.4 Hz, 1P), 16.04 (d, J=27.7 Hz, 1P).

### Synthesis of 1-(3-amino-2-hydroxypropyl)-3-(2-carboxy-2-hydroxy-2-phosphonoethyl)pyridinium (4b)

Compound 1b (0.52 g, 2.10 mmol, 1.00 eq), 2-hydroxy-2-phosphono-3-pyridin-3-ylpropanoic acid, was dissolved in 10 mL water, and the pH adjusted to 5.9 with 1 M NaOH. To this solution, 0.45 g of 5 (2.57 mmol, 1.22 eq) in minimal MeOH was added. The reaction mixture was stirred at 50° C. for 6 h and then stirred at RT overnight, yielding 90% of 2b (³¹P NMR). The solvent was removed in vacuo, and the residue was washed with diethyl ether, filtered, and dried in a desiccator, leaving 2b (diastereoisomeric mixture), which was then used without further purification. ¹H NMR (D₂O): δ 8.53-8.49 (brd, 1H), 8.47 (d, J=6.0 Hz, 1H), 8.29-8.24 (m, 1H), 7.78 (dd, J=8.3 Hz, 6.2 Hz, 1H), 4.64-4.58 (brd, 1H), 4.27-4.19 (m, 1H), 4.00-3.91 (m, 1H), 3.49-3.43 (m, 1H), 3.23-3.00 (m, 3H), 1.27 (s, 9H). ³¹P NMR (D₂O): δ 14.97 (s, 1P).

The entire sample of 2b was dissolved in 50:50 water:TFA (v/v). After stirring at RT for 4 h, a 100% yield of 4b was obtained according to ¹H NMR. The solvent was then removed in vacuo, and the residue was washed with diethyl ether, filtered, and dried, yielding 4b (a diastereoisomeric mixture) as white crystals, which were used without further purification. 1H NMR (D₂O): δ 8.68-8.64 (m, 1H), 8.63-8.59 (m, 1H), 8.42-8.39 (m, 1H), 7.91 (dd, J=8.0 Hz, 6.4 Hz, 1H), 4.78-4.72 (m, 1H), 4.46-4.33 (m, 1H), 4.24-4.14 (m, 1H), 3.59-3.49 (m, 1H), 3.33-3.21 (m, 2H), 2.95 (ddd, J=13.3, 10.0, 3.6 Hz, 1H). ³¹P NMR (D₂O): δ 12.73-12.51 (m, 1P).

### Synthesis of 1-(3-amino-2-hydroxypropyl)-3-(2,2-diphosphonoethyl)pyridinium (4c)

Compound 1c (38.0 mg, 0.14 mmol, 1.00 eq), (2-pyridin-3-ylethane-1,1-diyl)bis(phosphonic acid), was dissolved in 1 mL water and the pH adjusted to 5.4 with 1 M NaOH. To this solution was added 25.5 mg of 5 (0.15 mmol, 1.07 eq) in minimal MeOH. The reaction mixture was stirred at 40° C. overnight, and the reaction was monitored by ³¹P NMR. After 19 h, 80% of 2c yielded. Thus, an additional 5.30 mg (0.03 mmol, 0.21 eq) of 5 in MeOH was added to the reaction mixture. After 42 h, 90% of the desired product was obtained. The solvent was removed in vacuo, and the resulting white powder was washed with diethyl ether, filtered, and dried, giving 2c, which was used without further purification. ¹H NMR (D₂O): δ 8.69 (s, 1H), 8.49 (d, J=6.1 Hz, 1H), 8.42 (d, J=8.3 Hz, 1H), 7.84 (dd, J=8.1 Hz, 6.1 Hz, 1H), 4.66-4.61 (m, 1H), 4.27 (dd, J=13.5 Hz, 9.6 Hz, 1H), 4.00-3.94 (m, 1H), 3.30-3.10 (m, 4H), 2.15 (tt, J=21.0 Hz, 7.2 Hz, 1H), 1.26 (s, 9H). ³¹P NMR (D₂O): δ 17.25 (s, 2P).

The entire sample of 2c was dissolved in 50:50 water:TFA (v/v). After stirring at RT for 4 h, a 100% yield of 4c was obtained according to ¹H NMR. The solvent was removed in vacuo, and the residue was washed with diethyl ether and methanol, filtered, and dried, yielding 4c as white crystals, which was then used without further purification. ¹H NMR (D₂O): δ 8.73 (s, 1H), 8.54 (d, J=6.1 Hz, 1H), 8.45 (d, J=8.2 Hz, 1H), 7.89 (dd, J=8.1 Hz, 6.1 Hz, 1H), 4.76-4.70 (m, 1H), 4.37 (dd, J=13.4 Hz, 9.3 Hz, 1H), 4.26 (t, J=9.6 Hz, 1H), 3.37-3.13 (m, 3H), 2.98 (dd, J=13.1, 9.8 Hz, 1H), 2.28 (tt, J=21.4, 7.2 Hz, 1H). ³¹P NMR (D₂O): δ 17.35 (s, 2P).

### Synthesis of Drug-Linker Intermediate 3a

Route B:

Compound 1a (57 mg, 0.2 mmol, 1 eq.) (1-hydroxy-1-phosphono-2-pyridin-3-yl-ethyl)phosphonic acid) was dissolved in 4 mL of D₂O and pH was adjusted to 6.0 with Na₂CO₃ (s). To this solution was added epichlorohydrin 6 (79 μL, 1 mmol, 5 eq.). The reaction mixture was stirred at rt. The reaction progress was monitored by ³¹P and ¹H NMR. In 4 h no more epichlorohydrin remained in the reaction mixture. About 8% of the unreacted starting material and no O-alkylation by-products were observed by ³¹P NMR. ¹H NMR (400 MHz, D₂O) δ 8.72 (s, 1H), 8.49 (d, J=6.2 Hz, 1H), 8.44 (d, J=8.0 Hz, 1H), 7.80 (dd, J=8.0, 6.1 Hz, 1H), 4.74 (dd, J=13.5, 2.9 Hz, 1H), 4.47 (dd, J=13.6, 9.3 Hz, 1H), 4.29 (dtd, J=9.3, 4.7, 2.9 Hz, 1H), 3.77-3.56 (m, 2H), 3.29 (t, J=11.6 Hz, 2H).

### Synthesis of drug-linker intermediate 4d (3-(3-amino-2-hydroxypropyl)-1-(2-hydroxy-2,2-diphosphonoethyl)-1H-imidazol-3-ium)

Route A:

Compound 1d (40.0 mg, 0.15 mmol, 1.00 eq.), [1-hydroxy-2-(1H-imidazol-1-yl)ethane-1,1-diyl]bis(phosphonic acid), was dissolved in 3 mL water and the pH adjusted to 7.4 with Na₂CO₃ (s). To this solution was added 51 mg of 5 (0.29 mmol, 2 eq) in minimal MeOH. The reaction mixture was stirred at 50° C. overnight, and the reaction was monitored by ³¹P NMR. After 19 h, 76% of 2d yielded. Thus, an additional 10.9 mg (0.06 mmol, 0.42 eq) of 5 in MeOH was added to the reaction mixture. After 41 h, less than 10% of starting materials was left and 81% of desired compound 2d yielded with ˜15% of side products. The solvent was removed in vacuo, and the resulting white powder was washed with diethyl ether, filtered, and dried, giving 2d, which was used without further purification. The entire sample of 2d was dissolved in 50:50 water:TFA (v/v). After stirring at RT for overnight, Boc group was fully deprotected, and desired compound 4d was obtained according to ¹H NMR. The solvent was removed in vacuo, and the residue was washed with diethyl ether and methanol, filtered, and dried, which was then subjected to SAX HPLC purification. SAX column (Macherey-Nagel 21.4 mm×250 mm SP15/25 Nucleogel column), flow rate: 9 mL/min, UV-VIS detection at 230 nm. Sample was eluted with A: H₂O, B: 0.5 M TEAB pH 7.5 using a gradient that was increased from 0-30% over 10 min, maintained at 30% from 10-15 min, and then increased to 100% of buffer B from 15-35 min. The biggest peak eluting from 12-14 min was collected (the retention time has ±1.5 min error between different runs), and solvents were evaporated, yielding compound 4d for next step reaction. ¹H NMR (D₂O): δ 8.76 (s, 1H), 7.45 (s, 1H), 7.33 (s, 1H), 4.53 (dd, J=7.4, 6.8 Hz, 2H), 4.33 (d, J=13.3 Hz, 1H), 4.11-4.07 (m, 2H), 3.21-3.17 (m, 1H), 2.89 (brd, 1H). ³¹P NMR (D₂O): δ 14.02 (s, 2P).

Route B:

Compound 1d (50.0 mg, 0.18 mmol, 1.00 eq.), [1-hydroxy-2-(1H-imidazol-1-yl)ethane-1,1-diyl]bis(phosphonic acid), was dissolved in 4 mL water and the pH adjusted to 7.4-7.8 with Na₂CO₃ (s). To this solution was added 72.8 μL of 6 (0.93 mmol, 5 eq). The reaction mixture was stirred at RT overnight, and the reaction was monitored by ³¹P NMR. After 19 h, 79% of 3d yielded with ˜15% of side products and less than 10% of starting materials was left. The solution of reaction mixture was washed with diethyl ether (3×), and the solvent of aqueous phase was removed in vacuo, giving 3d, which was used without further purification. The entire sample of 3d was dissolved in 2 mL of NH₃.H₂O. After stirring at RT for 30 hrs, chlorine was displaced and desired compound 4d was obtained according to MS. The solvent was removed in vacuo, and the residue was washed with diethyl ether and methanol, filtered, and dried, which was then subjected to SAX HPLC purification. SAX column (Macherey-Nagel 21.4 mm×250 mm SP15/25 Nucleogel column), flow rate: 9 mL/min, UV-VIS detection at 230 nm. Sample was eluted with A: H₂O, B: 0.5 M TEAB pH 7.5 using a gradient that was increased from 0-30% over 10 min, maintained at 30% from 10-15 min, and then increased to 100% of buffer B from 15-35 min. The biggest peak eluting from 13.5-15.0 min was collected (the retention time has ±1.5 min error between different runs), and solvents were evaporated, yielding compound 4d for next step reaction. ¹H NMR (D₂O): δ 8.76 (s, 1H), 7.45 (s, 1H), 7.33 (s, 1H), 4.53 (dd, J=7.4, 6.8 Hz, 2H), 4.33 (d, J=13.3 Hz, 1H), 4.11-4.07 (m, 2H), 3.21-3.17 (m, 1H), 2.89 (brd, 1H). ³¹P NMR (D₂O): δ 13.9 (s, 2P).

### Synthesis of drug-linker intermediates 4e (1-(3-amino-2-hydroxypropyl)-3-(2-hydroxy-2,2-diphosphonoethyl)imidazo[1,2-a]pyridin-1-ium, Route B)

Compound 1e (140.4 mg, 0.44 mmol, 1.00 eq), (1-Hydroxy-2-imidazo[1,2-a]pyridin-3-yl-1-phosphonoethyl)phosphonic acid, was dissolved in 4 mL water and the pH adjusted to 7.4-7.8 with 10 M NaOH. To this solution was added 170.9 μL of 6 (2.18 mmol, 5 eq). The reaction mixture was stirred at RT overnight, and the reaction was monitored by ³¹P NMR. After 19 h, 70% of 3e yielded with ˜15% of side products and ˜15% of starting materials was left. The solution of reaction mixture was washed with diethyl ether (3-5 times), and the solvent of aqueous phase was removed in vacuo, giving 3e, which was used without further purification. The entire sample of 3e was dissolved in 8 mL of NH₃.H₂O. After stirring at RT for 44 hrs, chlorine was displaced and desired compound 4e was obtained according to MS. The solvent was removed in vacuo, and the residue was washed with diethyl ether and methanol, filtered, and dried, which was then subjected to SAX HPLC purification. SAX column (Macherey-Nagel 21.4 mm×250 mm SP15/25 Nucleogel column), flow rate: 9 mL/min, UV-VIS detection at 280 nm. Sample was eluted with A: H₂O, B: 0.5 M TEAB pH 7.6 using a gradient that was increased from 0-30% over 10 min, maintained at 30% from 10-18 min, and then increased to 100% of buffer B from 18-35 min. The biggest peak eluting from 9.4-11.5 min was collected (the retention time has ±1.5 min error between different runs), and solvents were evaporated, yielding compound 4e for next step reaction. ¹H NMR (D₂O): δ 8.77 (d, J=7.0 Hz, 1H), 7.87-7.70 (m, 3H), 7.32 (dt, J=7.8, 4.2 Hz, 1H), 4.47 (d, J=13.5 Hz, 1H), 4.40-4.13 (m, 2H), 3.53 (t, J=11.3 Hz, 2H), 3.29-3.20 (m, 1H), 2.99-2.90 (part. obscured by triethylamine, about 1H). ³¹P NMR (D₂O): δ 16.6 (s, 2P).

### Synthesis of drug-linker intermediates 4f (1-(3-amino-2-hydroxypropyl)-3-(2-carboxy-2-hydroxy-2-phosphonoethyl)imidazo[1,2-a]pyridin-1-ium, Route B)

Compound 1f (100 mg, 0.35 mmol, 1.00 eq), 2-hydroxy-3-imidazo[1,2-a]pyridin-3-yl-2-phosphonopropionic acid, was dissolved in 2.85 mL water and the pH adjusted to 7.4-7.8 with 10 M NaOH. To this solution was added 137 μL of 6 (1.75 mmol, 5 eq). The reaction mixture was stirred at RT overnight, and the reaction was monitored by ³¹P NMR. After 19 h, 61% of 3f yielded with ˜24% of side products and ˜15% of starting materials was left. The solution of reaction mixture was washed with diethyl ether (3-5 times), and the solvent of aqueous phase was removed in vacuo, giving 3f, which was used without further purification. The entire sample of 3f was dissolved in 5 mL of NH₃.H₂O. After stirring at RT for 44 hrs, chlorine was displaced and desired compound 4f was obtained according to MS. The solvent was removed in vacuo, and the residue was washed with diethyl ether and methanol, filtered, and dried, which was then subjected to SAX HPLC purification. SAX column (Macherey-Nagel 21.4 mm×250 mm SP15/25 Nucleogel column), flow rate: 9 mL/min, UV-VIS detection at 280 nm. Sample was eluted with A: H₂O, B: 0.5 M TEAB pH 7.6 using a gradient that was increased from 0-30% over 10 min, maintained at 30% from 10-18 min, and then increased to 100% of buffer B from 18-35 min. The biggest peak eluting from 9.3-11.5 min was collected (the retention time has ±1.5 min error between different runs), and solvents were evaporated, yielding compound 4f for next step reaction. ¹H NMR (D₂O): δ 8.64 (d, J=7.1 Hz, 1H), 7.91-7.71 (m, 2H), 7.62 (s, 1H), 7.29 (ddd, J=7.0, 5.9, 2.2 Hz, 1H), 4.51-4.39 (m, 1H), 4.35-4.14 (m, 2H), 3.66 (dd, J=15.8, 3.6 Hz, 1H), 3.35 (dd, J=15.7, 7.4 Hz, 1H), 3.26-3.15 (m, 1H), 2.98-2.79 (m, 1H). ³¹P NMR (D₂O): δ 14.8.

General Method for Preparation of Compounds 7a-7f

The following synthesis and purification steps were performed under minimal lighting. 4a-f (3-5 eq) were dissolved in H₂O. The pH was adjusted to 8.3 with solid Na₂CO₃. 5(6)-FAM, SE (1 eq), 5(6)-RhR-X, SE (1 eq), 5(6)-ROX, SE (1 eq) (commercially available 5(6)-ROX, SE (mixed isomer) includes 5-ROX, SE: 6-ROX, SE: and bisSE at the ration of 98:1:1 according to the characteristic data provided by Invitrogen; after purification at this step, the minor components were separated and the final products are the 5-isomers), AF647, SE (1 eq, the structure of AF647 was determined by MS and ¹H NMR of its corresponding BP/PC conjugates, which is different from the proposed structure in literature [55]), sulfo-Cy5, SE (1 eq) or IRDye 800CW, SE (1 eq) was dissolved in anhydrous DMF and combined with the water solution. The pH was re-adjusted to 8.2-8.5 with Na₂CO₃, dissolving any precipitate, and the reaction mixture stirred for 3 h—overnight under RT in darkness. Crude products of FAM and 5(6)-RhR-X conjugates (7a1-7a4, 7b1-7b2, 7c1-7c2, 7d1-7d2, 7e1-7e2, 7f1-7f2) were purified by TLC on plates 20×20 cm or 7×20 cm (the size of TLC plates chosen depends on the total crude amount) eluted with 100% MeOH. Crude reaction mixtures of other dye conjugates were not purified by TLC. The phosphonate-containing compounds remaining at the origin (R_{f}=0) were extracted with water; the combined aqueous extracts may be treated with Chelex (sodium form) to aid the extraction process. The solution was centrifuged, and then concentrated in vacuo. The resulting solids were then dissolved in either water, 20% MeOH in 0.1 M triethylammonium acetate buffer (TEAAc, pH 5.0-5.5) or triethylammonium carbonate buffer (TEAC, pH 7.0-7.8) and filtered through Nanosep 30K Omega filters. The solution was then purified by preparative/semi-preparative reverse-phase HPLC according to the appropriate method. The final amount of labeled product was calculated from the UV-VIS absorption spectrum, and the isolated eluent concentrated in vacuo and lyophilized.

Method A:

Dynamax C18 (21.4 mm×25 cm, 5 μm, 100 Å pore size) column, flow rate 8.0 mL/min, UV detection at 260 nm, gradient as follows: linearly increasing from 10% MeOH 0.1 M TEAAc (pH 5.0-5.5) or TEAC (pH 7.0-7.8, buffer A) to 40% of 75% MeOH 0.1 M TEAAc (pH 5.0-5.5) or TEAC (pH 7.0-7.8, buffer B) in 12 min, then increasing to 70% of buffer B from 12-100 min;

Method B:

Dynamax C18 (21.4 mm×25 cm, 5 μm, 100 Å pore size) column, flow rate 8.0 mL/min, UV detection at 260 nm, isocratic elution with 20% MeOH 0.1 M TEAC (pH 7.0-7.8, buffer A) for 12 min, linearly increasing to 100% of 70% MeOH 0.1 M TEAC (pH 7.0-7.8, buffer B) from 12-22 min;

Method C:

Beckman Ultrasphere ODS C18 (250×10 mm, 5 μm, 80 Å pore size), flow rate 6.0 mL/min, UV detection at 260 nm and 568 nm, isocratic elution of 20% MeOH in 0.1 M TEAC (pH 7.0-7.8, buffer A) for 5 min, linearly increasing to 100% of 75% MeOH in 0.1 M TEAC (pH 7.0-7.8, buffer B) in 1 min;

Method D:

Beckman Ultrasphere ODS C18 (250×10 mm, 5 μm, 80 Å pore size), flow rate 4.0 mL/min, UV detection at 260 nm and 568 nm, isocratic elution of 20% MeOH in 0.1 M TEAAc (pH 5.0-5.5, buffer A) for 5 min, linearly increasing to 100% of 75% MeOH in 0.1 M TEAAc (pH 5.0-5.5, buffer B) in 1 min;

Method E:

Beckman Ultrasphere ODS C18 (250×10 mm, 5 μm, 80 Å pore size), flow rate 4 mL/min, UV detection at 260 nm and 568 nm, isocratic elution of 20% MeOH in 0.1 M TEAC (pH 7.0-7.8, buffer A) for 5 min, linearly increasing to 100% of 70% MeOH in 0.1 M TEAC (pH 7.0-7.8, buffer B) in 1 min;

Method F:

Beckman Ultrasphere ODS C18 (250×10 mm, 5 μm, 80 Å pore size), flow rate 4.0 mL/min, UV detection at 260 nm and 576 nm, isocratic elution of 20% MeOH in 0.1 M TEAAc buffer (pH 5.0-5.5, buffer A) for 5 min, linearly increasing to 100% of 70% MeOH in 0.1 M TEAAc buffer (pH 5.0-5.5 buffer B) in 5 min;

Method G:

Beckman Ultrasphere ODS C18 (250×10 mm, 5 μm, 80 Å pore size), flow rate 4.0 mL/min, UV detection at 260 nm and 576 nm, isocratic elution of 10% MeOH in 0.1 M TEAC buffer (pH 7.0-7.8, buffer A) for 5 min, linearly increasing to 100% of 70% MeOH in 0.1 M TEAC buffer (pH 7.0-7.8, buffer B) in 5 min;

Method H:

Beckman Ultrasphere ODS C18 (250×10 mm, 5 μm, 80 Å pore size), flow rate 4.0 mL/min, UV detection at 260 nm (7a6, 7b4) or 230 nm (7d3) and 598 nm, isocratic elution of 20% MeOH in 0.1 M TEAAc buffer (pH 5.0-5.5, buffer A) for 5 min, linearly increasing to 40% of 70% MeOH in 0.1 M TEAAc buffer (pH 5.0-5.5, buffer B) in 20 min;

Method I:

Beckman Ultrasphere ODS C18 (250×10 mm, 5 μm, 80 Å pore size), flow rate 4.0 mL/min, UV detection at 230 nm (7d1-7d2) or 280 nm (7e1, 7e2, 7f1, 7f2) and 492 nm, gradient as follows: linearly increasing from 10% MeOH in 0.1 M TEAC (pH 7.0-7.8, buffer A) to 40% of 75% MeOH in 0.1 M TEAC (pH 7.0-7.8, buffer B) in 25 min, then increasing to 70% of buffer B from 25-100 min;

Method J:

Beckman Ultrasphere ODS C18 (250×10 mm, 5 μm, 80 Å pore size), flow rate 4.0 mL/min, UV detection at 230 nm and 598 nm, isocratic elution of 20% MeOH in 0.1 M TEAAc buffer (pH 5.0-5.5, buffer A) for 7 min, linearly increasing to 100% of 70% MeOH in 0.1 M TEAAc buffer (pH 5.0-5.5, buffer B) from 7-25 min;

Method K:

Beckman Ultrasphere ODS C18 (250×10 mm, 5 μm, 80 Å pore size), flow rate 4.0 mL/min, UV detection 230 nm and 598 nm, isocratic elution of 20% MeOH in 0.1 M TEAAc buffer (pH 5.0-5.5, buffer A) for 5 min, linearly increasing to 40% of 70% MeOH in 0.1 M TEAAc buffer (pH 5.0-5.5, buffer B) from 5-15 min; maintained at 40% of buffer B from 15-30 min, finally increase to 100% of buffer B from 30-35 min.

### 5(6)-FAM-RIS (7a1), 5-FAM-RIS (7a2): 1-(3-(3-carboxy-4-(6-hydroxy-3-oxo-3H-xanthen-9-yl)benzamido)-2-hydroxypropyl)-3-(2-hydroxy-2,2-diphosphonoethyl)pyridin-1-ium; 6-FAM-RIS (7a3): 1-(3-(4-carboxy-3-(6-hydroxy-3-oxo-3H-xanthen-9-yl)benzamido)-2-hydroxypropyl)-3-(2-hydroxy-2,2-diphosphonoethyl)pyridin-1-ium)

Synthesized according to the method above with 86.5 mg of 4a (as TFA⁻, Na⁺ salt, 0.18 mmol, 1.7 eq) in 2 mL of H₂O and pH adjusted to 8.3 with Na₂CO₃ (s), to which 50.0 mg of 5(6)-FAM, SE (0.11 mmol, 1.00 eq) in 600 μL anhydrous DMF was added; the pH of reaction solution was further adjusted to pH 8.3 to dissolve precipitates, and the reaction mixture was then stirred at RT for overnight. After TLC purification (100% MeOH as eluent), the product was purified by HPLC according to Method A with TEAAc buffers. Peaks eluting from 25-75 min were collected. During evaporation of the buffer solution, product precipitated from the solution. Consequently, a second HPLC purification was performed according to Method A but eluting with TEAC buffers (pH 7.5). Obtained 29.0 mg, 46.8% yield (triethylammonium bicarbonate salt). ¹H NMR (D₂O): δ 8.76-8.62 (m, 1H), 8.54-8.48 (m, 1H), 8.42 (dt, J=17.6, 7.8 Hz, 1H), 8.04 (s 0.6H), 7.92-7.64 (m, 2H), 7.45 (s, 0.4H), 7.13 (s, 1H), 6.93 (ddd, J=9.1, 4.5, 1.9 Hz, 2H), 6.45-6.38 (m, 4H), 4.82-4.70 (m, 1H), 4.44-4.28 (m, 1H), 4.28-4.12 (m, 1H), 3.65-3.55 (m, 1H), 3.56-3.44 (m, 1H), 3.44-3.19 (m, 2H). ³¹P NMR (D₂O): δ 16.36 (s, 2P).

HPLC Separation of 5- and 6-FAM-RIS (7a2 and 7a3):

Synthesized according to method described for 7a1. Under HPLC conditions described as Method A, 6-FAMRIS and 5-FAMRIS elute at very different retention times, 27 and 44 min (the retention time has ±1.5 min error between different runs), respectively. Each isomer was collected separately and then concentrated in vacuo to remove buffer. Compound 7a2 and 7a3 were also directly synthesized from 5-FAM, SE and 6-FAM, SE according to the method described above. Detailed NMR descriptions of 7a2 and 7a3 can be found from ref. [56].

### 5(6)-FAM-RISPC (7b1, also known as 5(6)-FAM−3-PEHPC. 5-FAM-RISPC: 3-(2-carboxy-2-hydroxy-2-phosphonoethyl)-1-(3-(3-carboxy-4-(6-hydroxy-3-oxo-3H-xanthen-9-yl)benzamido)-2-hydroxypropyl)pyridin-1-ium; 6-FAM-RISPC: 3-(2-carboxy-2-hydroxy-2-phosphonoethyl)-1-(3-(4-carboxy-3-(6-hydroxy-3-oxo-3H-xanthen-9-yl)benzamido)-2-hydroxypropyl)pyridin-1-ium)

Synthesized according to method described above with 94.8 mg of intermediate 4b (0.21 mmol, 3.5 eq) in 1 mL of water and pH adjusted to 8.3 with Na₂CO₃ (s), to which added in 30 mg of 5(6)-FAM, SE (0.06 mmol, 1.0 eq) in 200 μL anhydrous DMF. The pH of reaction solution was further adjusted to pH 8.4 to dissolve precipitates, and the reaction mixture was then stirred at RT for overnight. After TLC purification (100% MeOH as eluent), the mixture was purified by HPLC according to Method B. Peaks eluting at 21-25 min were collected together as 7b1. Obtained 23.2 mg, 53.9% yield (triethylammonium bicarbonate salt). ¹H NMR (D₂O): δ 8.66-8.44 (m, 2H), 8.29 (brd, 1H), 8.05 (s, 0.6H), 7.89-7.68 (m, 2H), 7.45 (s, 0.4H), 7.13 (d, J=8.0 Hz, 1H), 6.93 (d, J=9.2 Hz, 2H), 6.50-6.35 (m, 4H), 4.43-4.25 (m, 2H), 3.78-3.55 (m, 1H), 3.54-3.41 (m, 2H), 3.41-3.23 (m, 1H), 2.87 (part. obscured by triethylamine, about 1H). ³¹P NMR (D₂O): δ 15.15 (brs, 1P). HRMS (positive ion MALDI): calcd 679.1324 m/z; found [M]⁺=679.1321 m/z.

### 5(6)-FAM-dRIS (7c1, 5-FAM-dRIS: 1-(3-(3-carboxy-4-(6-hydroxy-3-oxo-3H-xanthen-9-yl)benzamido)-2-hydroxypropyl)-3-(2,2-diphosphonoethyl)pyridin-1-ium; 6-FAM-dRIS: 1-(3-(4-carboxy-3-(6-hydroxy-3-oxo-3H-xanthen-9-yl)benzamido)-2-hydroxypropyl)-3-(2,2-diphosphonoethyl)pyridin-1-ium)

Synthesized according to method described above with 53 mg of intermediate 4c (0.1 mmol, 2.5 eq) in 1 mL HPLC water and pH adjusted to 8.3 with Na₂CO₃ (s), to which added in 18.0 mg of 5(6)-FAM, SE (0.04 mmol, 1.00 eq) in 100 μL anhydrous DMF. The pH of reaction solution was further adjusted to pH 8.4 to dissolve precipitates, and the reaction mixture was then stirred at RT for overnight. After TLC purification, the mixture was purified according to Method B. Peaks eluting from 27-45 min were collected as 7c1. Obtained 9.4 mg, 36% yield (triethylammonium acetate salt). ¹H NMR (D₂O): δ 8.73-8.69 (m, 1H), 8.50 (d, J=6.1 Hz, 1H), 8.47-8.36 (m, 1H), 8.06 (d, J=1.9 Hz, 0.6H), 7.94-7.68 (m, 2H), 7.53 (s, 0.4H), 7.23 (d, J=8.0 Hz, 1H), 6.99 (dd, J=9.7, 2.4 Hz, 2H), 6.50-6.40 (m, 4H), 4.82-4.72 (m, 1H), 4.42-4.29 (m, 1H), 4.29-4.09 (m, 1H), 3.62 (dd, J=14.1, 4.5 Hz, 1H), 3.23-3.11 (obscured by solvent peak, about 1H), 3.58-3.32 (m, 2H), 2.14-1.87 (m, 1H). ³¹P NMR (D₂O): δ 17.17 (brs). HRMS (positive ion MALDI): calcd 699.1139 m/z; found [M]⁺=699.1137 m/z.

### 5(6)-RhR-RIS (7a4, 1-{3-[6-({4-[6-(diethylamino)-3-(diethylimino)-3H-xanthen-9-yl]-3-sulfobenzene}sulfonamido)hexanamido]-2-hydroxypropyl}-3-(2-hydroxy-2,2-diphosphonoethyl)pyridinium)

Synthesized according to method described above with 11.2 mg of compound 4a (0.032 mmol, 4.9 eq) in 0.5 mL H₂O and pH adjusted to 9.0 with Na₂CO₃ (s), to which 5 mg of RhR-X, SE (0.0065 mmol, 1 eq.) in 250 μL DMF was added. After TLC purification, the mixture was purified by HPLC according to Method C. Peak eluting between 12.8-18 minutes (the retention time has ±1.0 min error between different runs) were collected. Obtained 0.2 mg, 3% yield (as a triethylammonium bicarbonate salt). ¹H NMR (D₂O): δ 8.66 (s, 1H), 8.49-8.31 (m, 3H), 8.09 (s, 1H), 7.70 (s, 1H), 7.59-7.33 (m, 1H), 7.01-6.57 (m, 7H), 4.22-3.89 (m, 3H), 3.55-3.23 (m, obscured by solvent peak and TEA peak, around 12H), 3.02-2.96 (m, obscured by TEA peak, around 3H), 2.19-2.01 (m, 2H), 1.47-1.24 (m, obscured by TEA peak, around 5H), 1.10 (obscured by TEA peak, about 12H). ³¹P NMR (D₂O): δ 16.76 (s, 2P). HRMS (positive ion MALDI): calcd 1011.2913 m/z, found [M−H]⁺=1010.2866 m/z.

### 5(6)-RhR-RISPC (7b2, 3-(2-carboxy-2-hydroxy-2-phosphonoethyl)-1-{3-[6-({4-[6-(diethylamino)-3-(diethylimino)-3H-xanthen-9-yl]-3-sulfobenzene}sulfonamido)hexanamido]-2-hydroxypropyl}pyridinium, also known as 5(6)-RhR-3-PEHPC)

Synthesized according to method described above with 10.9 mg of compound 4b (0.04 mmol, 3 eq) in 0.5 mL of H₂O and pH adjusted to 8.3 with Na₂CO₃ (s), to which 5 mg of 5(6)-RhR-X, SE in 500 μL DMF was added. After TLC purification, the solution was then purified by HPLC according to Method D. Peak eluting at 13 min (the retention time has ±1.0 min error between different runs) was collected as 7b2. Obtained 2.1 mg, 33% yield (triethylammonium bicarbonate salt). ¹H NMR (400 MHz, D₂O): δ 8.51 (s, 1H), 8.43 (d, J=10.1 Hz, 2H), 8.29 (s, 1H), 8.09 (d, J=8.0 Hz, 1H), 7.83-7.66 (m, 1H), 7.45 (d, J=8.0 Hz, 1H), 6.87-6.70 (m, 4H), 6.65 (s, 2H), 4.56-4.43 (m, 1H), 4.16 (d, J=14.4 Hz, 1H), 3.95 (s, 1H), 3.48 (dd, J=23.6, 8.0 Hz, 8H), 3.28-3.12 (m, obscured by solvent, about 4H), 2.93 (td, J=17.6, 16.8, 8.9 Hz, 3H), 2.10 (t, J=7.6 Hz, 2H), 1.47-1.21 (m, 5H), 1.09 (obscured by TEA peak, about 12H). ³¹P NMR (D₂O): 15.2 (s). HRMS (positive ion MALDI): calcd 975.3148 m/z, found [M−H]⁺⁼974.3118 m/z.

### 5(6)-RhR-dRIS (7c2, 1-{3-[6-({4-[6-(diethylamino)-3-(diethylimino)-3H-xanthen-9-yl]-3-sulfobenzene}sulfonamido)hexanamido]-2-hydroxypropyl}-3-(2,2-diphosphonoethyl)pyridin-1-ium)

Synthesized according to method described above with 9.4 mg of compound 4c (0.02 mmol, 3.3 eq) in 0.6 mL H₂O and pH adjusted to 8.3 with Na₂CO₃ (s), to which 5 mg of 5(6)-RhR-X, SE (0.0065 mmol, 1 eq) in 0.45 mL of DMF was added. Precipitation was observed. The reaction mixture was stirred for 2 h, then evaporated to dryness. The resulting solids were extracted with acetone (3×1 mL, in order to remove partially unconjugated dye. The remaining precipitate was dissolved in ˜2 mL H₂O and purified by TLC as described above, followed by HPLC purification (Method E). A broad peak eluting between 13-17.3 min (the retention time has ±1.0 min error between different runs) was collected. Obtained 2.75 mg, 42.5% yield (triethylammonium bicarbonate salt). ¹H NMR (D₂O): δ 8.67 (d, J=2.2 Hz, 1H), 8.50-8.36 (m, 3H), 8.10 (t, J=6.9 Hz, 1H), 7.79 (dd, J=8.4, 6.4 Hz, 1H), 7.42 (t, J=8.6 Hz, 1H), 6.86-6.68 (m, 4H), 6.68-6.57 (m, 2H), 4.62-4.50 (m, 1H), 4.19 (dd, J=13.3, 9.7 Hz, 1H), 4.10-3.94 (m, 1H), 3.45 (p, J=7.0 Hz, 8H), 3.34-3.13 (m, 4H), 2.97 (q, J=6.7 Hz, 3H), 2.36-2.01 (m, 3H), 1.53-1.23 (m, 5H), 1.10 (td, J=7.0, 3.2 Hz, 12H). ³¹P NMR (D₂O): 17.29 (s). HRMS (positive ion MALDI): calcd 995.2695 m/z, found [M−H]⁺=994.2872 m/z.

### 5(6)-ROX-RIS (7a5, 5-ROX-RIS: 16-[2-carboxy-4-({2-hydroxy-3-[4-(2-hydroxy-2,2-diphosphonoethyl)pyridin-1-ium-1-yl]propyl}carbamoyl)phenyl]-3-oxa-9λ⁵,23-diazaheptacyclo[17.7.1.1^{5,9}.0^{2,17}.0^{4,15}.0^{23,27}.0^{13,28}]octacosa-1(27),2(17),4,9(28), 13,15,18-heptaen-9-ylium)

Synthesized according to method described above with 23.6 mg of compound 4a (0.047 mmol, 3 eq.) in 0.8 mL of H₂O/NaHCO₃ (pH 9.0), to which 10 mg of 5(6)-ROX, SE (0.016 mmol, 1 eq.) in 200 μL anhydrous DMF was added, and the solution stirred overnight. The solvent was concentrated under vacuo, and the resulting purple residue was dissolved in 20% MeOH in 0.1 M TEAAc buffer (pH 5.3) and purified by HPLC (Method F). Peaks eluting at 17.0 min (the retention time has ±1.0 min error between different runs) were collected as 7a5. Obtained 7.4 mg, 54.0% yield (triethylammonium acetate salt). ¹H NMR (D₂O): δ 8.74 (s, 1H), 8.55 (d, J=6.0 Hz, 1H), 8.43 (d, J=8.1 Hz, 1H), 8.07 (s, 1H), 7.81 (t, J=7.2 Hz, 1H), 7.63 (d, J=7.6 Hz, 1H), 6.77 (d, J=7.9 Hz, 1H), 6.52 (s, 2H), 4.35-4.21 (m, 2H), 3.57-3.48 (m, 2H), 3.37-3.16 (m, 13H), 2.70-2.62 (m, 2H), 2.47-2.27 (m, 5H), 1.79-1.53 (m, 7H). ³¹P NMR (D₂O): 16.36 (s). HRMS (positive ion MALDI): calcd 873.2660 m/z, found [M−H]⁺=873.2647 m/z.

### 5(6)-ROX-RISPC (7b3, also known as 5(6)-ROX-3-PEHPC, 5-ROX-RISPC: 16-[2-carboxy-4-({3-[4-(2-carboxy-2-hydroxy-2-phosphonoethyl)pyridin-1-ium-1-yl]-2-hydroxypropyl}carbamoyl)phenyl]-3-oxa-9λ⁵,23-diazaheptacyclo[17.7.1.1^{5,9}.0^{2,17}.0^{4,15}.0^{23,27}.0^{13,28}]octacosa-1(27),2(17),4,9(28), 13,15,18-heptaen-9-ylium)

Synthesized according to method described above with 54.3 mg of 4b (0.119 mmol, 3 eq.) in 1.6 mL of H₂O/NaHCO₃ (pH 9.0) and 25 mg of 5(6)-ROX, SE (0.04 mmol, 1 eq.) in 1 mL anhydrous DMF, and the solution was stirred overnight. The solvent was concentrated under vacuo, and the resulting purple residue was dissolved in 10% MeOH in 0.1 M TEAC buffer (pH 7.0) and purified by HPLC (Method G). Peaks eluting at 21.9 min (the retention time has ±1.0 min error between different runs) were collected as 7b3. Obtained 9.4 mg, 35.0% yield (triethylammonium bicarbonate salt). ¹H NMR (D₂O): δ 8.63 (s, 1H), 8.54 (d, J=6.2 Hz, 1H), 8.31 (s, 1H), 8.03 (d, J=1.9 Hz, 1H), 7.86-7.78 (m, 1H), 7.75 (d, J=8.0 Hz, 1H), 7.00 (s, 1H), 6.59 (s, 2H), 4.45-4.33 (m, 1H), 4.31-4.18 (m, 1H), 3.67-3.48 (m, 2H), 3.36-3.14 (m, 13H), 2.83-2.72 (m, 2H), 2.51-2.35 (m, 5H), 1.81-1.62 (m, 7H). ³¹P NMR (D₂O): 14.34 (s). HRMS (negative ion MALDI): calcd 835.2750 m/z, found [M−3H]⁻=835.2733 m/z.

### AF647-RIS (7a6, 2-(5-(3-(6-((2-hydroxy-3-(3-(2-hydroxy-2,2-diphosphonoethyl)pyridin-1-ium-1-yl)propyl)amino)-6-oxohexyl)-3-methyl-5-sulfo-1-(3-sulfopropyl)indolin-2-ylidene)penta-1,3-dien-1-yl)-3,3-dimethyl-5-sulfo-1-(3-sulfopropyl)-3H-indol-1-ium)

Synthesized according to method described above with 25.9 mg of compound 4a (0.05 mmol, 10 eq.) in 1 mL of H₂O/NaHCO₃ (pH 8.3) and 5 mg of AF647, SE (0.005 mmol, 1 eq.) in 250 μL anhydrous DMF, and the solution was stirred at RT overnight. The solvent was concentrated under vacuo, and the resulting blue residue was dissolved in 20% MeOH in 0.1 M TEAAc buffer (pH 5.3) and purified by HPLC (Method H). Peaks eluting at 19.8 min (the retention time has ±1.0 min error between different runs) were collected as 7a6. Obtained 4.8 mg, 76.7% yield (triethylammonium acetate salt). ¹H NMR (D₂O): δ 8.61 (s, 1H), 8.45 (d, J=6.2 Hz, 1H), 8.39 (d, J=8.1 Hz, 1H), 8.00 (t, J=13 Hz, 2H), 7.80-7.67 (m, 5H), 7.31-7.20 (m, 2H), 6.55 (t, J=12.6 Hz, 1H), 6.37-6.23 (m, 2H), 4.70-4.60 (obscured by HDO, about 1H), 4.14-3.98 (m, 4H), 2.92-2.80 (m, 6H), 2.14-2.10 (m, 5H), 1.95-1.92 (m, 2H), 1.57-1.53 (m, 9H), 1.48-1.45 (m, 1H), 1.29-1.27 (m, 2H), 1.00-0.95 (m, 3H), 0.82-0.79 (m, 3H), 0.45-0.43 (m, 2H). ³¹P NMR (D₂O): δ 16.50 (d, J=26.9 Hz, 1P), 16.30 (d, J=29.0 Hz, 1P). HRMS (positive ion MALDI): calcd 1198.2410 m/z, found [M−H]⁺=1197.2358 m/z.

### AF647-RISPC (7b4, 2-(5-(3-(6-((3-(3-(2-carboxy-2-hydroxy-2-phosphonoethyl)pyridin-1-ium-1-yl)-2-hydroxypropyl)amino)-6-oxohexyl)-3-methyl-5-sulfo-1-(3-sulfopropyl)indolin-2-ylidene)penta-1,3-dien-1-yl)-3,3-dimethyl-5-sulfo-1-(3-sulfopropyl)-3H-indol-1-ium, also known as AF647-3-PEHPC)

Synthesized according to method described above with 22.5 mg of compound 4b (0.05 mmol, 10 eq.) in 1 mL of H₂O and pH adjusted to 8.3 with Na₂CO₃ (s), to which 5 mg of AF647, SE (0.005 mmol, 1 eq.) in 300 μL anhydrous DMF was added. The solution was stirred at RT overnight. The solvent was concentrated under vacuo, and the resulting blue residue was dissolved in 20% MeOH in 0.1 M TEAAc buffer (pH 5.3) and purified by HPLC (Method H). Peaks eluting at 18.8 min (the retention time has ±1.0 min error between different runs) were collected as 7b4. Obtained 5.3 mg, 87.2% yield (triethylammonium acetate salt). ¹H NMR (D₂O): δ 8.47 (m, 2H), 8.25 (d, J=7.7 Hz, 1H), 7.94 (t, J=13.1 Hz, 2H), 7.81-7.73 (m, 1H), 7.73-7.63 (m, 4H), 7.26 (t, J=8.0 Hz, 2H), 6.52 (t, J=12.4 Hz, 1H), 6.25 (dd, J=13.6, 9.8 Hz, 2H), 4.70-4.60 (obscured by HDO, about 1H), 4.21-4.11 (m, 4H), 3.42-3.40 (m, 1H), 2.91-2.83 (m, 5H), 2.20-2.01 (m, 6H), 1.95-1.92 (m, 2H), 1.51-1.50 (m, 9H), 1.25-1.20 (obscured by triethylamine peak, about 4H), 0.99-0.95 (obscured by triethylamine peak, 4H), 0.69-0.42 (m, about 2H). ³¹P NMR (D₂O): δ 15.21 (s). HRMS (positive ion MALDI): calcd 1162.2645 m/z, found [M−H]⁺=1161.2572 m/z.

### 5-FAM-ZOL (7d1, 3-(3-(3-carboxy-4-(6-hydroxy-3-oxo-3H-xanthen-9-yl)benzamido)-2-hydroxypropyl)-1-(2-hydroxy-2,2-diphosphonoethyl)-1H-imidazol-3-ium) and 6-FAM-ZOL (7d2, 3-(3-(4-carboxy-3-(6-hydroxy-3-oxo-3H-xanthen-9-yl)benzamido)-2-hydroxypropyl)-1-(2-hydroxy-2,2-diphosphonoethyl)-1H-imidazol-3-ium)

Synthesized according to method described above with 60.2 mg of compound 4d (as TEA⁺ salt (4 eq. of TEA), 0.08 mmol, 2.7 eq.) in 0.5 mL of H₂O and pH adjusted to 8.4 with Na₂CO₃ (s), to which 15.2 mg of 5(6)-FAM, SE (0.03 mmol, 1 eq.) in 100 μL anhydrous DMF was added. The pH was adjusted to 8.3 to dissolve precipitates and the solution stirred at RT overnight. After TLC purification, the product was purified by HPLC (Method I). 6-FAM-ZOL (7d2) and 5-FAM-ZOL (7d1) were eluted at very different retention times, 20 and 30 min (the retention time has ±1.5 min error between different runs), respectively. Each isomer was collected separately and then concentrated in vacuo to remove buffer. Compound 7d1 and 7d2 could also be directly synthesized from 5-FAM, SE and 6-FAM, SE according to the method described above. Detailed NMR descriptions given below correspond to the HPLC-separated products. Total amount of 7d1 and 7d2 is 15.4 mg, 68.3% yield. 5-FAM-ZOL (7d1, triethylammonium bicarbonate salt): obtained 9.2 mg (triethylammonium bicarbonate salt). ¹H NMR (D₂O): δ 8.74 (s, 1H), 8.11-8.03 (m, 1H), 7.84 (dd, J=8.0, 1.9 Hz, 1H), 7.45 (t, J=1.7 Hz, 1H), 7.34 (t, J=1.8 Hz, 1H), 7.21 (d, J=7.9 Hz, 1H), 6.99 (d, J=9.0 Hz, 2H), 6.50-6.43 (m, 4H), 4.57-4.44 (m, 2H), 4.36 (d, J=12 Hz, 1H), 4.22-4.03 (m, 2H), 3.57 (dd, J=14.0, 4.5 Hz, 1H), 3.43 (dd, J=14.0, 6.7 Hz, 1H). ³¹P NMR (D₂O): δ 14.02 (s). HRMS (positive ion MALDI): calcd 704.1041 m/z, found M⁺=704.1013 m/z. 6-FAM-ZOL (7d2, triethylammonium bicarbonate salt): obtained 6.2 mg (triethylammonium bicarbonate salt). ¹H NMR (D₂O): δ 8.70 (s, 1H), 7.90 (dd, J=8.1, 1.8 Hz, 1H), 7.78 (d, J=8.1, 1H), 7.57 (d, J=1.7 Hz, 1H), 7.43 (t, J=1.7 Hz, 1H), 7.30 (t, J=1.8 Hz, 1H), 7.03 (d, J=8.8 Hz, 2H), 6.56-6.42 (m, 4H), 4.56-4.42 (m, 2H), 4.32 (d, J=12.5 Hz, 1H), 4.17-3.99 (m, 2H), 3.51 (dd, J=14.1, 4.2 Hz, 1H), 3.40-3.33 (m, 1H). ³¹P NMR (D₂O): δ 14.03 (s). HRMS (positive ion MALDI): calcd 704.1041 m/z, found M⁺=704.1027 m/z.

### AF647-ZOL (7d3, 2-(5-(3-(6-((2-hydroxy-3-(1-(2-hydroxy-2,2-diphosphonoethyl)-1H-imidazol-3-ium-3-yl)propyl)amino)-6-oxohexyl)-3-methyl-5-sulfo-1-(3-sulfopropyl)indolin-2-ylidene)penta-1,3-dien-1-yl)-3,3-dimethyl-5-sulfo-1-(3-sulfopropyl)-3H-indol-1-ium)

Synthesized according to method described above with 18.9 mg of compound 4d (0.05 mmol, 5 eq.) in 500 μL of H₂O and pH adjusted to 8.4 with Na₂CO₃ (s), to which 10 mg of AF647, SE (0.0105 mmol, 1 eq.) in 300 μL anhydrous DMF was added. The solution was stirred at RT overnight and then was concentrated under vacuo, and the resulting blue residue was dissolved in 20% MeOH in 0.1 M TEAAc buffer (pH 5.3) and purified by HPLC (Method H). Peaks eluting at 16.5 min were collected as 7d3 (the retention time has ±1.5 min error between different runs). Obtained 6.6 mg, 53.1% yield (triethylammonium acetate salt). ¹H NMR (D₂O): δ 8.63 (s, 1H), 7.99 (t, J=13.2 Hz, 2H), 7.78-7.65 (m, 4H), 7.39 (s, 1H), 7.34-7.20 (m, 3H), 6.55 (t, J=12.5 Hz, 1H), 6.29 (dd, J=13.6, 9.7 Hz, 2H), 4.52-4.48 (m, 2H), 4.26-4.07 (m, 5H), 3.98-3.81 (m, 2H), 2.95-2.85 (m, 5H), 2.13-2.08 (m, 6H), 1.93-1.89 (m, 2H), 1.58-1.54 (m, 9H), 1.34-1.21 (m, 3H), 1.04-0.94 (m, 2H), 0.81-0.66 (m, 1H), 0.52-0.36 (m, 1H). ³¹P NMR (D₂O): δ 13.52 (s). HRMS (positive ion MALDI): calcd 1186.2290 m/z, found [M−H]⁺=1186.2337 m/z.

### 800CW-ZOL (7d4, (E)-2-((E)-2-(3-((E)-2-(1-(6-((2-hydroxy-3-(1-(2-hydroxy-2,2-diphosphonoethyl)-1H-imidazol-3-ium-3-yl)propyl)amino)-6-oxohexyl)-3,3-dimethyl-5-sulfo-3H-indol-1-ium-2-yl)vinyl)-2-(4-sulfophenoxy)cyclohex-2-en-1-ylidene)ethylidene)-3,3-dimethyl-1-(4-sulfonatobutyl)indoline-5-sulfonate, sodium salt)

Synthesized according to method described above with 7.4 mg of compound 4d (0.021 mmol, 5.3 eq.) in 1 mL of H₂O and pH adjusted to 8.4 with Na₂CO₃ (s), to which 5 mg of IRDye 800CW, SE (0.004 mmol, 1 eq.) in 100 μL anhydrous DMF was added. The solution was stirred at 4° C. overnight and was then concentrated under vacuo, and the resulting greenish black residue was dissolved in 20% MeOH in 0.1 M TEAAc buffer (pH 5.3) and purified by HPLC (Method J). Peaks eluting at 23.5 min were collected as 7d4 (the retention time has ±1.5 min error between different runs). Obtained 4.8 mg, 83.2% yield (triethylammonium acetate salt). ¹H NMR (D₂O): δ 8.65 (s, 1H), 7.67 (d, J=8.6 Hz, 2H), 7.63-7.50 (m, 6H), 7.39 (s, 1H), 7.28 (t, J=1.8 Hz, 1H), 7.14-6.96 (m, 4H), 5.99-5.84 (dd, J=14.2, 9.4 Hz, 2H), 4.56-4.46 (m, 2H), 4.18 (d, J=12.6 Hz, 1H), 4.02-3.66 (m, 6H), 3.15-3.09 (m, 2H), 2.81-2.73 (m, 3H), 2.45 (brd, 5H), 2.09-2.06 (m, 2H), 1.83 (obscured by solvent peak, around 12H), 1.80-1.59 (obscured by solvent peak, around 6H), 1.53-1.38 (m, 4H). ³¹P NMR (D₂O): δ 13.65 (s). HRMS (positive ion MALDI): calcd 1330.2865 m/z, found [M−H]⁺=1330.2885 m/z.

### Sulfo-Cy55-ZOL (7d5, 1-(6-((2-hydroxy-3-(1-(2-hydroxy-2,2-diphosphonoethyl)-1H-imidazol-3-ium-3-yl)propyl)amino)-6-oxohexyl)-3,3-dimethyl-2-((1E,3E,5E)-5-(1,3,3-trimethyl-5-sulfonatoindolin-2-ylidene)penta-1,3-dien-1-yl)-3H-indol-1-ium-5-sulfonate, sodium salt)

Synthesized according to method described above with 22.71 mg of compound 4d (0.066 mmol, 5.1 eq.) in 0.95 mL of H₂O and pH adjusted to 8.34 with Na₂CO₃ (s), to which 10 mg of Sulfo-Cy5, SE (0.013 mmol, 1 eq.) in 450 μL anhydrous DMF was added. Precipitates could be seen. The solution was stirred at RT overnight and then was concentrated under vacuo, and the resulting blue residue was dissolved in 20% MeOH in 0.1 M TEAAc buffer (pH 5.3) and purified by HPLC (Method K). Peaks eluting at 18 min were collected as 7d5 (the retention time has ±1.5 min error between different runs). Obtained 5.3 mg, 41.2% yield (triethylammonium acetate salt). ¹H NMR (D₂O): δ 8.65 (s, 1H), 7.81 (td, J=13.1, 4.3 Hz, 2H), 7.74-7.56 (m, 4H), 7.39 (s, 1H), 7.26 (s, 1H), 7.15 (dd, J=8.4, 2.2 Hz, 2H), 6.32 (t, J=12.5 Hz, 1H), 6.00 (dd, J=19.6, 13.7 Hz, 2H), 4.57-4.44 (m, 2H), 4.17 (d, J=13.0 Hz, 1H), 3.92 (m, 4H), 3.42 (s, 3H), 2.16-2.09 (m, 2H), 1.68-1.61 (m, 2H), 1.53-1.41 (m, 15H), 1.26-1.17 (obscured by triethylamine peak, around 3H). ³¹P NMR (D₂O): δ 13.51 (s). MS (negative ion ESI): calcd 483.6 m/z, found [M−4H]²=484.0 m/z.

### 5-FAM-MIN (7e1, 1-(3-(3-carboxy-4-(6-hydroxy-3-oxo-3H-xanthen-9-yl)benzamido)-2-hydroxypropyl)-3-(2-hydroxy-2,2-diphosphonoethyl)imidazo[1,2-a]pyridin-1-ium) and 6-FAM-MIN (7e2, 1-(3-(4-carboxy-3-(6-hydroxy-3-oxo-3H-xanthen-9-yl)benzamido)-2-hydroxypropyl)-3-(2-hydroxy-2,2-diphosphonoethyl)imidazo[1,2-a]pyridin-1-ium)

Synthesized according to method described above with 22.4 mg of compound 4e (0.057 mmol, 2.5 eq.) in 1 mL of H₂O and pH adjusted to 8.58 with Na₂CO₃ (s), to which 10.8 mg of 5(6)-FAM, SE (0.023 mmol, 1 eq.) in 300 μL anhydrous DMF was added. The pH was adjusted to 8.4 to dissolve precipitates and the solution was stirred at RT overnight. After TLC purification, the product was purified by HPLC (Method I). 6-FAM-MIN (7e2) and 5-FAM-MIN (7e1) were eluted at very different retention times, 21.5 and 31.5 min (the retention time has ±3 min error between different runs), respectively. Each isomer was collected separately and then concentrated in vacuo to remove buffer. Compound 7e1 and 7e2 could also be directly synthesized from 5-FAM, SE and 6-FAM, SE according to the method described above. Detailed NMR descriptions given below correspond to the HPLC-separated products. Total amount of 7e1 and 7e2 is 11.6 mg, 67.2% yield. 5-FAM-MIN (7e1, triethylammonium bicarbonate salt): obtained 6.3 mg (triethylammonium bicarbonate salt). ¹H NMR (D₂O): δ 8.77 (d, J=7.0 Hz, 1H), 8.09 (d, J=1.6 Hz, 1H), 7.94-7.67 (m, 4H), 7.31 (td, J=6.4, 1.6 Hz, 1H), 7.26 (d, J=8.0 Hz, 1H), 7.06 (s, 1H), 7.03 (s, 1H), 6.55 (dq, J=5.0, 2.3 Hz, 4H), 4.57-4.46 (m, 1H), 4.42-4.21 (m, 2H), 3.72-3.42 (m, 4H). ³¹P NMR (D₂O): δ 16.52 (s). HRMS (positive ion MALDI): calcd 754.1198 m/z, found M⁺=754.1178 m/z. 6-FAM-MIN (7e2, triethylammonium bicarbonate salt): obtained 5.3 mg (triethylammonium bicarbonate salt). ¹H NMR (D₂O): δ 8.75 (d, J=7.0 Hz, 1H), 7.89 (dd, J=8.1, 1.8 Hz, 1H), 7.82-7.67 (m, 4H), 7.55 (d, J=1.6 Hz, 1H), 7.27 (td, J=6.7, 1.6 Hz, 1H), 7.07 (s, 1H), 7.05 (s, 1H), 6.63-6.51 (m, 4H), 4.50-4.42 (m, 1H), 4.34-4.19 (m, 2H), 3.63-3.48 (m, 3H), 3.42 (dd, J=14.1, 6.9 Hz, 1H). ³¹P NMR (D₂O): δ 16.52 (s). HRMS (positive ion MALDI): calcd 754.1198 m/z, found M⁺=754.1187 m/z.

### 5-FAM-MINPC (7f1, also known as 5-FAM−3-IPEHPC, 3-(2-carboxy-2-hydroxy-2-phosphonoethyl)-1-(3-(3-carboxy-4-(6-hydroxy-3-oxo-3H-xanthen-9-yl)benzamido)-2-hydroxypropyl)imidazo[1,2-a]pyridin-1-ium) and 6-FAM-MINPC (7f2, also known as 6-FAM-3-IPEHPC, 3-(2-carboxy-2-hydroxy-2-phosphonoethyl)-1-(3-(4-carboxy-3-(6-hydroxy-3-oxo-3H-xanthen-9-yl)benzamido)-2-hydroxypropyl)imidazo[1,2-a]pyridin-1-ium)

Synthesized according to method described above with 20 mg of compound 4f (0.056 mmol, 2.5 eq.) in 1 mL of H₂O and pH adjusted to 8.57 with Na₂CO₃ (s), to which 10.5 mg of 5(6)-FAM, SE (0.022 mmol, 1 eq.) in 300 μL anhydrous DMF was added. The pH was adjusted to 8.4 to dissolve precipitates and the solution was stirred at RT overnight. After TLC purification, the product was purified by HPLC (Method I). 6-FAM-MINPC (7f2) and 5-FAM-MINPC (7f1) were eluted at very different retention times, 19 and 28 min (the retention time has ±1.5 min error between different runs), respectively. Each isomer was collected separately and then concentrated in vacuo to remove buffer. Compound 7f1 and 7f2 could also be directly synthesized from 5-FAM, SE and 6-FAM, SE according to the method described above. Detailed NMR descriptions given below correspond to the HPLC-separated products. Total amount of 7f1 and 7f2 was 11.7 mg, 73.2% yield. 5-FAM-MINPC (7f1, triethylammonium bicarbonate salt): obtained 6.3 mg (triethylammonium bicarbonate salt). ¹H NMR (D₂O): δ 8.69 (d, J=6.9 Hz, 1H), 8.08 (dq, J=1.6, 0.8 Hz, 1H), 7.88-7.75 (m, 3H), 7.68 (d, J=4.4 Hz, 1H), 7.33 (ddd, J=6.9, 5.8, 2.1 Hz, 1H), 7.27 (dt, J=8.0, 0.7 Hz, 1H), 7.01 (dt, J=9.2, 0.9 Hz, 2H), 6.53-6.46 (m, 3H), 4.50 (dt, J=14.6, 2.9 Hz, 1H), 4.32 (dd, J=14.6, 9.0 Hz, 1H), 4.26 (dq, J=9.2, 5.1, 4.1 Hz, 1H), 3.72 (dd, J=15.9, 3.2 Hz, 1H), 3.62 (ddd, J=14.1, 4.8, 1.9 Hz, 1H), 3.48 (ddd, J=14.0, 7.0, 4.3 Hz, 1H), 3.40 (dd, J=15.7, 7.4 Hz, 1H). ³¹P NMR (D₂O): δ 14.82 (s). HRMS (positive ion MALDI): calcd 718.1433 m/z, found M⁺=718.1399 m/z. 6-FAM-MINPC (7f2, triethylammonium bicarbonate salt): obtained 5.4 mg (triethylammonium bicarbonate salt). ¹H NMR (D₂O): δ 8.64 (d, J=7.0 Hz, 1H), 7.85 (ddd, J=8.1, 1.8, 0.9 Hz, 1H), 7.78-7.62 (m, 4H), 7.50 (dt, J=1.7, 0.8 Hz, 1H), 7.28 (td, J=6.8, 1.6 Hz, 1H), 7.04-6.95 (m, 2H), 6.53-6.45 (m, 4H), 4.46-4.37 (m, 1H), 4.29-4.12 (m, 2H), 3.68 (dd, J=15.7, 3.6 Hz, 1H), 3.52 (dd, J=14.0, 4.0 Hz, 1H), 3.43-3.33 (m, 2H). ³¹P NMR (D₂O): δ 15.03 (s). HRMS (positive ion MALDI): calcd 718.1433 m/z, found M⁺⁼718.1416 m/z.

Investigation of Reaction of Pyridine with Epichlorohydrin (6)

### Pyridine:Epichlorohydrin (1:5 Ratio of Equivalent) in D₂O:

Pyridine (16 μL, 0.2 mmol, 1 eq.) was dissolved in 4 mL of D₂O and pH was brought to 6.2 using methylenebisphosphonic acid (s). To this solution epichlorohydrin (79 μL, 1 mmol, 5 eq.) was added. The reaction progress was monitored by ¹H NMR. In 4 h no more epichlorohydrin remained in the reaction mixture. The formation of two products was observed with the ration of A:B=1:2, with about 20% of unreacted pyridine remaining.

Pyridine:epichlorohydrin (1:1 ratio of equivalent) in D₂O:

Pyridine (16 μL, 0.2 mmol, 1 eq.) was dissolved in 4 mL of D₂O and pH was brought to 6.2 using methylenebisphosphonic acid (s). To this solution epichlorohydrin (16 μL, 0.2 mmol, 1 eq.) was added. The reaction progress was monitored by ¹H NMR. In 4 h no more epichlorohydrin remained in the reaction mixture. The formation of two products was observed with the ration of A:B=6:1, with about 54% of unreacted pyridine remaining).

### Pyridine:Epichlorohydrin (1:5 Ratio of Equivalent) in MeCN:

Pyridine (16 μL, 0.2 mmol, 1 eq.) was dissolved in 4 mL of acetonitrile. To this solution epichlorohydrin (79 μL, 1 mmol, 5 eq.) was added. In 4 h all volatiles were evaporated and ¹H NMR of the residue was taken. Compound B was observed as the only product. 1H NMR (400 MHz, D₂O): δ 8.92 (d, J=5.8 Hz, 2H, ring), 8.66 (t, J=7.6 Hz, 1H, ring), 8.20-8.08 (m, 2H, ring), 5.19 (dd, J=14.5, 2.2 Hz, 1H, CH—N), 4.60 (dd, J=14.5, 7.4 Hz, 1H, CH—N), 3.70 (m, 1H, CHOCH2), 3.13 (t, J=4.0 Hz, 1H, CHOCH2), 2.83 (dd, J=4.0, 2.8 Hz, 1H, CHOCH2).

¹H NMR reported by Demberelnyamba: ¹H NMR (500 mHz, D₂O): 8.92-8.85 (m, 2H, ring), 8.66-8.59 (m, 1H, ring), 8.16-8.10 (m, 2H, ring), 5.11-4.66 (m, 1H, CHOCH₂), 3.80-3.63 (m, 2H, CHOCH₂), 1.2-1.17 (d, 2H, CH₂—N). FAB-MS (MeOH matrix): m/z 135.9 [100%, GlPyb].

### UV-VIS Absorption and Fluorescence Emission Spectra

All labeled samples were dissolved in water and diluted with 0.1 M phosphate buffer or 1×PBS (for 7d5). Assuming that the labeled bisphosphonates have the same extinction coefficient (E) as the carboxylic acid of the free fluorescent label, the final concentrations for all labeled products were calculated from UV-VIS absorption spectra at λ=493 nm (ε=73000 M⁻¹ cm⁻¹ at pH 7.2) for FAM conjugates (7a1-7a3, 7b1, 7c1, 7d1-7d2, 7e1-7e2, 7f1-7f2), λ=567.5 nm (ε=114850 M⁻¹cm⁻¹ at pH 7.5) for RhR-X conjugates (7a4, 7b2, 7c2), λ=580 nm (c=72000 M⁻¹cm⁻¹ at pH 8.0) for ROX conjugates (7a5, 7b3), λ=648 nm (ε=240000 M⁻¹cm⁻¹ at pH 7.2) for AF647 conjugates (7a6, 7b4, 7d3), λ=774 nm (ε=240000 M⁻¹cm⁻¹ at pH 7.4) for 800CW conjugates (7d4), λ=644 nm (ε=271000 M⁻¹cm⁻¹ at pH 7.4, 1×PBS) for Sulfo-Cy5 conjugates (7d5) [57].

Emission spectra for FAM and RhR-X conjugates were recorded using an excitation wavelength of 490 nm or 520 nm, respectively. Emission spectra for AF647 conjugates and Sulfo-Cy5 conjugates were recorded using an excitation wavelength of 600 nm. Emission spectra of 5(6)-ROX conjugates were recorded using an excitation wavelength of 575 nm. Emission spectra of IRDye CW800 conjugate was recorded using an excitation wavelength of 750 nm. The set-up of excitation slit, emission slit, integration time and increment were determined to get optimal spectra for each compound respectively, depending on the sample concentration and spectrometer used.

### Hydroxyapatite Column Chromatography Assay

The fast performance liquid chromatography (FPLC) system consisted of a Waters 650E advanced protein purification system (Millipore Corp., Waters chromatography division, Milford, Mass.), a 600E system controller and a 484 tunable absorbance detector for UV absorbance assessment. Ceramic hydroxyapatite [HAP, Caio(PO₄)6(OH)₂, Macro-Prep® Ceramic Hydroxyapatite Type II 20 μm 100 g, Bio-Rad Laboratories, Inc. Hercules, Calif.] was equilibrated with 1 mM phosphate buffer (pH 6.8) and packed in a 0.66 cm (diameter)×6.5 cm (length) glass column (Omnifit, Bio-chem Valve™ inc., Cambridge, U.K.), which was attached to the Waters 650E advanced protein purification system. Each sample was prepared in 1 mM potassium phosphate buffer, and 100 μL of 1 mM (0.1 μmol) sample was injected into the FPLC system. As a consequence, the compounds were absorbed and subsequently eluted at a flow rate of 2 ml/min by using a linear concentration gradient of phosphate from 1 to 1000 mM at pH 6.8. Fractions of each sample were collected in 80 tubes using an automated fraction collector (Gilson, France) and then used for subsequent ultraviolet (SPECTRAmax PLUS 384, Molecular Devices, CA) or fluorescence spectrometry detection (WALLAC VICTOR™, 1420 MULTILABEL COUNTER, Perkin Elmer, USA). Each fraction contained eluent in 0.3 min. The elution profile for each sample was determined in triplicate for statistical analysis (Prism, GraphPad Software, USA). Using the native BP (RIS, ZOL and MIN) as a retention time control/comparator, the chromatographic profiles of each fluorescent probe were normalized to its BP counterpart to allow relative comparisons of separations performed at different times. Data are presented as mean retention times normalized to native BP±standard deviation (SD).

### Quantitative Measurement of BP-HAP Interaction by Using Langmuir Adsorption Isotherms

1 mM phosphate-buffered saline (PBS) with 0.15 M NaCl (pH 6.8) was prepared freshly. Stock solutions of 5(6)-ROX-RIS, 5(6)-ROX-RISPC, AF647-RIS and AF647-RISPC were made by dissolving the compounds in 1 mM PBS to yield a 10 mM solution. Hydroxyapatite (Macro-Prep® Ceramic hydroxyapatite Type II 20 μM 100 g) was obtained from Bio-Rad Laboratories, Inc. Hercules, Calif.

To measure and compare the bone mineral affinities of fluorescent BP/PCs, adsorption isotherm studies were carried out under identical experimental conditions. Accurately weighed HAP powder (1.4-1.6 mg) was suspended in 4 mL clear vial containing the appropriate volume of 1 mM PBS with 0.15 M NaCl (pH 6.8) for 3 hours. After premixing, 10 mM fluorescent BP stock solutions were added, resulting in concentrations of the fluorescent BP/PC additives ranging as 25, 50, 100, 200 and 300 μM. Equilibrium with the HAP was performed by rotating the vials end-over-end on a shaker at room temperature for 16 hours. Each sample was prepared in triplicate. Subsequent to the equilibrium period, the vials were centrifuged at 10,000 rpm for 5 min to separate the solids and the supernatant. 0.3 mL of the supernatant was collected and the equilibrium solution concentration was measured by using Nanodrop UV spectrometer. For the calibration series, fluorescent BP/PC standards were prepared by serial dilution from the stock solution with the same isotherm buffer to give the range from 0 to 400 μM. Calibration curves were constructed using standard solutions of the target fluorescent BP.

The amount of fluorescent BP/PC bound to the HAP (mol/m²) was calculated by comparing the end point concentration of fluorescent BP/PC detected after equilibrium to the initial fluorescent BP/PC additive concentration (M) using the following equation:

Fluorescent BP/PC HAP surface concentration=(Initial fluorescent BP/PC concentration−end point concentration)/HAP surface area of the sample, where HAP surface area of the sample was 6700 m²/L in our case. A plot of fluorescent BP/PC HAP surface concentration versus BP end point concentration provided the adsorption isotherm.

To describe the equilibrium binding of fluorescent BP/PC to HAP as a function of increasing fluorescent BP/PC concentration, the experimental data were fitted to a saturation binding equation: Y (specific binding)=Bmax*X/(K_{d}+X) by using a non-linear curve-fitting algorithm, implemented in the Prism program (Graphpad, USA). Where X is the concentration of the fluorescent BP/PC, Y is the specific binding, and Bmax is the maximum number of binding sites, expressed in the same units as the Y-axis. K_{d} is the equilibrium dissociation constant, expressed in the same units as the X-axis (concentration). When the drug concentration equals K_{d}, half of the binding sites are occupied at equilibrium.

### Inhibition of Protein Prenylation and Cell Viability Assays

To determine the effect of fluorescent BP probes on protein prenylation, J774.2 mouse macrophages were plated out at 2×10⁵ cells/mL in 24-well plates and left to adhere overnight. Cells were then treated with 10 or 100 μM of fluorescent analogues of RIS, dRIS, and ZOL, the respective native BP, or vehicle, for 24 h. Cells were lysed in radioimmunoprecipitation buffer, and proteins were separated by sodium dodecyl sulfate polyacrylamide gel electrophoresis and transferred to polyvinyl difluoride membranes by western blotting. Membranes were then incubated with antibodies to the unprenylated form of Rap1A (uRap1A) and the housekeeping protein β-actin, which were detected by incubation with fluorescently-labeled secondary antibodies and scanning of membranes on a LI-COR Infrared Imager. Results shown are representative of 2 independent experiments. The ratio between abundance of unprenylated RaplA and β-actin is indicated for each sample below the blots in FIG. 5.

To determine the effect of fluorescent BP analogues on cell viability, J774.2 mouse macrophages were plated at 2×10⁵ cells/mL in 96-well plates and left to adhere overnight. Cells were then treated with 10, 100 or 500 μM of fluorescent analogues of RIS, dRIS, and ZOL, the respective native BP, or vehicle, for 48 h. At the end of the incubation period, AlamarBlue reagent was added to each well and incubation continued for a further 3 h. Fluorescence was detected on a plate reader, and expressed as percent of vehicle control. Results are shown as mean±SD of ≧2 independent experiments, performed at least in duplicate.

### Synthesis of bifunctional azido-containing N-heterocyclic bisphosphonate (amino-azido-para-dRIS, 7, 1-(3-amino-2-azidopropyl)-4-(2,2-diphosphonoethyl)pyridin-1-ium)

### Synthesis of para-dRIS (23, tetraisopropyl (2-(pyridin-4-yl)ethane-1,1-diyl)bis(phosphonate))

To NaH (420 mg, 60% in oil, 9.63 mmol, 1.1 eq.) in 10 mL of dry DMF was added 4-(chloromethyl)pyridine-HCl (1.43 g, 8.71 mmol, 1 eq.) in 15 mL of dry DMF at 0° C. with stirring under N₂. In another flask, to 1.03 g of NaH (60% in oil, 25.78 mmol, 3 eq.) dispersed in 15 mL of dry THF was added tetraisopropyl methylenebisphosphonate (6.0 g, 17.42 mmol, 2 eq.) drop-wise at 0° C. under N₂, and stirring was continued at 0° C. for 30-45 min then for 1 h at room temperature. The 4-(chloromethyl)pyridine solution was added to the tetraisopropyl methylenebisphosphonate carbanion solution at 0° C. and stirred for 8 h at 70° C. The reaction was quenched by the addition of 100-200 μL of EtOH, cooled in freezer for 0.5 h, then dispersed in 100 mL of chilled H₂O, and extracted with chilled CH₂Cl₂ (100 mL×2). The organic CH₂Cl₂ phase was then dispersed in 150 mL of chilled 0.25 M HCl solution; shake well and the aqueous phase was collected, and further extracted by CHCl₃. The CHCl₃ phase was collected and dried over MgSO₄, and then concentrated to obtain 1.9 g of 23, 50% yield. ¹H NMR (D₂O): δ 8.56 (d, J=6.8 Hz, 2H), 7.88 (d, J=6.8 Hz, 2H), 4.62-4.52 (m, 4H), 3.33 (td, J=15.9, 7.1 Hz, 2H), 2.98 (tt, J=23.9, 7.1 Hz, 1H), 1.14 (ddd, J=25.2, 6.2, 1.4 Hz, 24H). ³¹P NMR (D₂O): δ 19.83 (s). MS: calcd 435.1 m/z, found [M+Na]⁺=458.1 m/z.

### Synthesis of para-dRIS-linker-OH (24, 4-(2,2-bis(diisopropoxyphosphoryl)ethyl)-1-(3-((tert-butoxycarbonyl)amino)-2-hydroxypropyl)pyridin-1-ium)

500 mg of para-dRIS (23, 1.15 mmol, 1 eq.) was dissolved in 2 mL of isopropanol in a pressure-tight glass vial. Then 100 μL of DIEA (0.57 mmol, 0.5 eq.) was added to the solution by syringe followed by adding linker 5 (790 mg, 4.56 mmol, 4 eq.) in 1 mL of isopropanol. The reaction mixture was stirred at 100° C. for 16 h. Solvent was removed under vaccuo and 12 mL of CHCl₃ was added in the reaction mixture, which was then dispersed in 3-4 mL of H₂O; shake well and collect the aqueous phase (repeat for 4-5 times). The aqueous phase was further washed by ether to remove excess linker 5, and re-extracted with CHCl₃. The final CHCl₃ phase was dried over MgSO₄, and then concentrated to obtain 520 mg of 24, 75% yield. ¹H NMR (CDCl₃): δ 9.38-9.27 (m, 2H), 7.83 (d, J=6.4 Hz, 2H), 6.14 (m, 2H), 5.09-4.99 (m, 1H), 4.80-4.64 (m, 5H), 4.21-4.04 (m, 1H), 3.39-3.19 (m, 4H), 2.50 (tt, J=23.6, 6.4 Hz, 1H), 1.36 (s, 9H), 1.28-1.22 (m, 24H). ³¹P NMR (CDCl₃): δ 18.73 (s). MS: calcd 609.3 m/z, found M⁺=609.1 m/z.

### Synthesis of para-dRIS-linker-N3-ester (26, 1-(2-azido-3-((tert-butoxycarbonyl)amino)propyl)-4-(2,2-bis(diisopropoxyphosphoryl)ethyl)pyridin-1-ium)

450 mg of para-dRisBP-linker-OH (24, 0.74 mmol, 1 eq.) was dissolved in 4 mL of anhydrous CH₂Cl₂ in a pressure-tight glass vial. Then 225 μL of TEA (1.6 mmol, 2.2 eq.) was added to the solution by syringe. Add 100 μL of MsCl (1.29 mmol, 1.7 eq.) drop-wise into the mixture at ice/water bath and stir the reaction mixture for 1.5 h until compound 24 was converted to intermediate 25 completely (monitored by MS and ³¹P NMR). Reaction mixture was briefly filtered and the filtrate was pumped to dryness under vacuo, quantitatively yielding brown oily intermediate 25.

350 mg of intermediate 25 (0.51 mmol, 1 eq.) was dissolved in 5 mL of anhydrous DMF, to which added in 330 mg NaN₃ (5.1 mmol, 10 eq.) and stir the reaction mixture vigorously at 50° C. oil bath for 30 hrs. The reaction was monitored by MS and ³¹P NMR.

The above mixture was filtered and the filtrate was concentrated under vacuo to give brown oil. 3 mL of CHCl₃ was used to dissolve the oil and filter off the insoluble solid. Remove the solvent of CHCl₃ and the residues were purified by silica column chromatography (R_{f}=0.3, CHCl₃/MeOH, 5:1). 220 mg of compound 26 was obtained, 70% yield. ¹H NMR (400 MHz, CDCl₃) δ 9.31 (s, 2H), 7.91 (s, 2H), 6.35 (s, 1H), 5.37 (s, 1H), 4.72 (dh, J=24.3, 6.3 Hz, 4H), 4.55-4.32 (m, 2H), 3.66-3.45 (m, 2H), 3.45-3.24 (m, 2H), 2.61 (t, J=25.3 Hz, 1H), 1.38 (s, 9H), 1.29-1.21 (m, 24H). ³¹P NMR (CDCl₃): δ 18.76 (s). MS (positive ion MALDI): calcd 634.3 m/z, found M⁺=634.1 m/z.

### Synthesis of amino-azido-para-dRIS (20, 1-(3-amino-2-azidopropyl)-4-(2,2-diphosphonoethyl)pyridin-1-ium)

50 mg of para-dRIS-linker-N3-ester (26, 0.08 mmol) was dissolved in 1 mL of CH₃CN followed by adding 0.3 mL of BTMS in a pressure-tight glass vial. Stir the mixture at r.t. for 24 hrs. Then the mixture was pumped under vacuo to dryness, which was added with 0.5 mL of methanol and stirred for 0.5 h. Remove the methanol to give the crude product for further HPLC purification. Preparative C18 column (Phenomenex Luna 5μ C18 column, 100 Å, 21.2 mm×250 mm, 5), flow rate: 8 mL/min, UV-VIS detection at 260 nm. Sample was eluted with A: 0.1 M triethylammonium bicarbonate (TEAB), pH 7.8, B: CH₃CN, using a gradient that was increased from 0-3% of eluent B over 20 min, and then increased to 100% of eluent B from 20-24 min followed by decreasing to 0% of eluent B from 24-25 min. The peak eluting at 17.6 min was collected (the retention time has ±1.0 min error between different runs), and solvents were evaporated, obtained 23 mg of compound 20, 80% yield. ¹H NMR (500 MHz, D₂O): δ 8.55 (d, J=6.6 Hz, 2H), 7.95 (d, J=6.6 Hz, 2H), 4.63 (d, J=3.2 Hz, 1H), 4.30 (dd, J=13.9, 9.7 Hz, 1H), 4.06-3.96 (m, 1H), 3.34-3.19 (m, 2H), 2.90 (d, J=4.5 Hz, 1H), 2.73 (dd, J=13.7, 7.4 Hz, 1H), 2.23-2.07 (tt, J=20.8, 7.3 Hz, 1H). ³¹P NMR (D₂O): δ 14.8. MS: calcd 366.1 m/z, found [M−2H]⁻=364.4 m/z, [M+Na]⁺=388.0, [M+2Na]⁺=410.0 m/z.

### Clickable Reactivity Studies of Amino-Azido-Para-dRIS (20)

### Synthesis of 5(6)-FAM-alkyne (29, 5-FAM-alkyne: 2-(6-hydroxy-3-oxo-3H-xanthen-9-yl)-5-(prop-2-yn-1-ylcarbamoyl)benzoic acid, 6-FAM-alkyne: 2-(6-hydroxy-3-oxo-3H-xanthen-9-yl)-4-(prop-2-yn-1-ylcarbamoyl)benzoic acid)

4.2 μL (0.065 mmol, 3 eq.) of propargylamine was added to a solution of 10.4 mg (0.022 mmol, 1 eq.) of 5(6)-carboxyfluorescein, succinimidyl ester in DMF (0.5 mL). After 5 h of stirring at r.t., the solvent was removed under vacuo and the crude mixture was purified by a silica gel TLC plate (R_{f}=0.7, acetone/CH₂Cl₂, 1:1) to give 7.7 mg (85%) of 5(6)-FAM-alkyne (29). ¹H NMR (500 MHz, Methanol-d₄): δ 8.98 (dt, J=1.5, 0.6 Hz, 1H), 8.73 (dd, J=8.0, 1.7 Hz, 1H), 8.70-8.62 (m, 2H), 8.18 (dt, J=1.3, 0.5 Hz, 1H), 7.86 (dt, J=8.0, 0.6 Hz, 1H), 7.28-7.20 (m, 7H), 7.11 (ddd, J=9.0, 6.9, 2.4 Hz, 4H), 4.76 (d, J=2.6 Hz, 2H), 4.64 (d, J=2.6 Hz, 2H), 3.19 (td, J=2.6, 0.4 Hz, 1H), 3.11 (td, J=2.5, 0.5 Hz, 1H). MS: calcd 413.1 m/z, found [M−H]r=412.3 m/z, [M+H]⁺=414.4 m/z.

### Synthesis of 5(6)-FAM-triazole-para-dRIS (30, 5-isomer: 1-(3-amino-2-(4-((3-carboxy-4-(6-hydroxy-3-oxo-3H-xanthen-9-yl)benzamido)methyl)-1H-1,2,3-triazol-1-yl)propyl)-4-(2,2-diphosphonoethyl)pyridin-1-ium; 6-isomer: 1-(3-amino-2-(4-((4-carboxy-3-(6-hydroxy-3-oxo-3H-xanthen-9-yl)benzamido)methyl)-1H-1,2,3-triazol-1-yl)propyl)-4-(2,2-diphosphonoethyl)pyridin-1-ium)

Dissolve 4.6 mg of amino-azido-para-dRIS (20, 12.6 μmol, 1.8 eq.) and 3 mg of 5(6)-FAM-alkyne (29, 7 μmol, 1 eq.) in water in a pressure-tight vial. Add 0.5 M triethylammonium bicarbonate (TEAB) buffer, pH 8.0, to final concentration as 0.2 M and volume of 1 mL. Add 28 μL of CuSO₄/sodium ascorbate solution (50 mM/75 mM in D₂O, 20% eq. Cu catalyst). Degas the solution by pumping and then flushing with argon, repeat three times. Split the solution into two halves. Incubate them at room temperature and 45° C. water bath overnight.

The reaction was quenched by adding Chelex resin followed by 2 h of ultrasonication. Then the mixture was monitored by TLC (eluent 100% methanol) which shows almost absence of reactant 5(6)-FAM-alkyne (29). Reaction mixture was then adjusted to pH 3.0 by 0.5 M HCl until no more precipitates formed. Precipitates were collected by centrifuging and then washed sequentially by acetone (0.5 mL×2) and diluted HCl (pH 3.0, 0.25 mL×2). Obtained 3 mg, 57% yield. ³¹P NMR spectrum indicates the purity of 5(6)-FAM-triazole-para-dRIS product (30) is >98%. MS: calcd 779.2 m/z, found [M−2H]⁻=777.5 m/z, [M+Na]⁺=801.3 m/z.

The 5- and 6-isomers were further separated by reverse phase HPLC. The precipitated product was re-dissolved in 0.1 M TEAB buffer and separated by semi-preparative reverse phase HPLC using the following conditions: Beckman Ultrasphere ODS C18 (250×10 mm, 5 μm, 80 Å pore size), flow rate 4.0 mL/min, UV detection at 256 nm and 370 nm, mobile phase: buffer A (0.1 M TEAB in 10% methanol, pH 8.0) and buffer B (0.1M TEAB in 75% methanol, pH 8.0). Gradient as follows: linearly increase from 0% of buffer B to 100% of buffer B in 20 min. 5- and 6-isomers were eluted at very different retention time, 7.2 min and 9.6 min. Collect each peak and remove solvent under vacuo. 5-isomer: ¹H NMR (400 MHz, D₂O): δ 8.26 (d, J=5.7 Hz, 2H), 7.99-7.75 (m, 5H), 7.58 (s, 1H), 7.06 (d, J=9.0 Hz, 2H), 6.64-6.47 (m, 4H), 5.25 (s, 1H), 5.13-4.89 (m, 3H), 3.47 (d, J=20.7 Hz, 2H), 3.26-3.17 (m, 1H), 3.01-2.87 (m, 2H), 2.19 (s, 1H). ³¹P NMR (400 MHz, D₂O): δ 17.05. 6-isomer: ¹H NMR (400 MHz, D₂O): δ 8.30 (d, J=6.6 Hz, 2H), 8.10 (d, J=1.7 Hz, 1H), 7.93 (s, 1H), 7.88 (t, J=6.3 Hz, 3H), 7.34 (d, J=8.0 Hz, 1H), 7.09 (d, J=9.2 Hz, 2H), 6.64-6.55 (m, 4H), 5.36 (s, 1H), 5.14 (d, J=10.0 Hz, 1H), 5.08-4.91 (m, 1H), 4.58 (d, J=2.4 Hz, 1H), 3.72-3.46 (m, 2H), 3.22 (s, 2H), 2.94 (d, J=7.6 Hz, 1H), 2.23 (s, 1H). ³¹P NMR (400 MHz, D₂O): δ 17.03.

### UV-VIS Absorption and Fluorescence Emission Spectra Measurement of Compound 30:

The 6- and 5-isomer of compound 30 were dissolved in water and diluted with 0.1 M phosphate buffer (pH 7.2). Assuming that two isomers of compound 30 have the same extinction coefficient (E) as the carboxylic acid of the free fluorescent label, the final concentrations for labeled products are calculated from UV-VIS absorption spectra at λ=493 nm (ε=73000 M⁻¹cm⁻¹ at pH 7.2).

### Synthesis of Fluorescently Labeled N-Heterocyclic Bisphosphonates Via Bisphosphonates Containing an Azido Group

### Synthesis of Glycidyl Azide 31

To prepare 31, the epichlorohydrin 6 (5 mmol) was dissolved in an aqueous solution of sodium azide (26.0 mmol in 8.0 mL), 4.6 mL acetic acid was then added and the solution was stirred for 5 h at 30° C. The solution was extracted with diethyl ether (3×8 mL). The combined organic phase was washed five times with 10 mL portions of sodium phosphate buffer (50 mM, pH 6.5). The organic phase was dried and the diethyl ether removed on a rotary evaporator to give 450 mg 1-azido-3-chloro-2-propanol (yield: 67%). Glycidyl azide 31 was prepared as an aqueous solution from 1-azido-3-chloro-2-propanol: 210 mg was added with 0.2 mL water and stirred, 1 M NaOH was then slowly added until the solution pH was stabilized at 12.5 for 5 min, 1 M HCl was then added to adjust the pH to 7.0, more water was added to make the volume 3.2 ml containing 0.5 M of glycidyl azide 31.

### Synthesis of the Azido-Containing N-Heterocyclic Bisphosphonate-Linker 32

Risedronate 1a (0.15 mmol) in 0.5 mL MES buffer (100 mM, pH 6.0) was added with 0.04 mL of 2M NaOH and 0.6 ml of glycidyl azide 31 (500 mM), sequentially. Solution was kept at RT overnight and then subjected to SAX-column HPLC purification, giving 65 mg white solid bisphosphonate-linker 32 (yield: 85.8%).

### Synthesis of Fluorescently Labeled N-Heterocyclic Bisphosphonate 34

Bisphosphonate-linker 32 (6.5 umol) and fluorescent labeled alkyne 33 (6.5 umol) in 0.5 ml triethylamine bicarbonate buffer (0.1 M, pH 8.0) was flushed by Ar and sealed in a pressure tight vial. Then 6.5 uL of CuSO₄/sodium ascorbate solution (50 mM/250 mM, 10% eq. Cu catalyst) was injected, the solution was kept at RT for 1 h and then purified by reverse phase HPLC to give 1.9 mg of triazole 34 (Yield: 50%).

### REFERENCES

The following publications are incorporated by reference herein in their entirety:
[1] a) R. G. G. Russell, N. B. Watts, F. H. Ebetino, M. J. Rogers, Osteoporos. Int. 2008, 19, 733-759; b) F. Ebetino, A. Hogan, S. Sun, M. Tsoumpra, X. Duan, J. Triffitt, A. Kwaasi, J. Dunford, B. Barnett, U. Oppermann, M. Lundy, A. Boyde, B. Kashemirov, C. McKenna, R. Russell, Bone 2011, 49 20-33.
[2] a) I. Reid, M. Bolland, A. Grey, Bone 2007, 41, 318-320; b) A. Siddiqi, A. Payne, S. Zafar, Oral Surg. Oral Med. Oral Pathol. Oral Radiol. Endod. 2009, 108, e1-8.
[3] a) I. Reid, Bone 2009, 44, 4-10; b) M. Allen, D. Burr, J Oral Maxillofac Surg 2009, 67, 61-70.
[4] S. Zhang, G. Gangal, H. Uludag, Chem. Soc. Rev. 2007, 36, 507-531.
[5] a) K. Bhushan, E. Tanaka, J. Frangioni, Angew. Chem. Int. Ed. 2007, 46, 7969-7971; b) J. Figueiredo, C. Passerotti, T. Sponholtz, H. Nguyen, R. Weissleder, J. Urol. 2008, 179, 1610-1614; c) F. Jaffer, P. Libby, R. Weissleder, Arterioscler. Thromb. Vasc. Biol. 2009, 29, 1017-1024; d) S. Subramanian, F. Jaffer, A. Tawakol, J. Nucl. Cardiol. 2010, 17, 135-144.
[6] V. Kubicek, I. Lukes, Future Med. Chem. 2010, 2, 521-531.
[7] S. Hilderbrand, R. Weissleder, Curr. Opin. Chem. Biol. 2010, 14, 71-79.
[8] S. Troyan, V. Kianzad, S. Gibbs-Strauss, S. Gioux, A. Matsui, R. Oketokoun, L. Ngo, A. Khamene, F. Azar, J. Frangioni, Ann. Surg. Oncol. 2009, 16, 2943-2952.
[9] S. Gioux, H. Choi, J. Frangioni, Mol. Imaging. 2010, 9, 237-255.
[10] F. Leblond, S. Davis, P. Valdes, B. Pogue, J. Photochem. PhotobiolB. 2010, 98, 77-94.
[11] a) K. Thompson, M. Rogers, F. Coxon, J. Crockett, Mol. Pharmacol. 2006, 69, 1624-1632; b) F. P. Coxon, K. Thompson, A. J. Roelofs, F. H. Ebetino, M. J. Rogers, Bone 2008, 42, 848-860.
[12] A. Zaheer, R. Lenkinski, A. Mahmood, A. Jones, L. Cantley, J. Frangioni, Nat. Biotechnol. 2001, 19, 1148-1154.
[13] B. A. Kashemirov, J. L. Bala, X. Chen, F. H. Ebetino, Z. Xia, R. G. G. Russell, F. P. Coxon, A. J. Roelofs, M. J. Rogers, C. E. McKenna, Bioconjug. Chem. 2008, 19, 2308-2310.
[14] M. Martin, W. Arnold, H. r. Heath, J. Urbina, E. Oldfield, Biochem. Biophys. Res. Commun. 1999, 263, 754-758.
[15] a) A. Hokugo, S. Sun, S. Park, C. E. McKenna, I. Nishimura, Bone 2013, 53, 59-68; b) S. Cheong, S. Sun, B. Kang, O. Bezouglaia, D. Elashoff, C. E. McKenna, T. L. Aghaloo, S. Tetradis, J. Oral Maxillofac. Surg. 2014; c) S. Bae, S. Sun, T. Aghaloo, J. E. Oh, C. E. McKenna, M. K. Kang, K. H. Shin, S. Tetradis, N. H. Park, R. H. Kim, Int. J. Mol. Med. 2014, 34, 559-563.
[16] W. S. Kang, S. Sun, K. Nguyen, B. A. Kashemirov, C. E. McKenna, S. A. Hacking, A. M. Quesnel, W. F. Sewell, M. J. McKenna, D. H. Jung, Otol. Neurotol. 2015, In press.
[17] S. Junankar, G. Shay, J. Jurczyluk, N. Ali, J. Down, N. Pocock, A. Parker, A. Nguyen, S. Sun, B. Kashemirov, C. E. McKenna, P. I. Croucher, A. Swarbrick, K. Weilbaecher, T. G. Phan, M. J. Rogers, Cancer discovery 2015, 5, 35-42.
[18] A. Hounslow, J. Carran, R. Brown, D. Rejman, G. Blackburn, D. Watts, J. Med. Chem. 2008, 51, 4170-4178.
[19] M. Baidya, F. Brotzel, H. Mayr, Org. Biomol. Chem. 2010, 8, 1929-1935.
[20] Note: pKa of ZOL and MIN were calculated by MarvinSketch and the error is +0.5. The concentration of microspecies was calculated according to the Henderson-Hasselbalch equation.
[21] D. Demberelnyamba, S. J. Yoon, H. Lee, Chem. Lett. 2004, 33, 560-561.
[22] E. R. van Beek, C. W. G. M. Lowik, F. H. Ebetino, S. E. Papapoulos, Bone 1998, 23, 437-442.
[23] X. Duan, PhD thesis, University of Oxford 2010.
[24] R. G. G. Russell, Z. Xia, J. E. Dunford, U. Oppermann, A. Kwaasi, P. A. Hulley, K. L. Kavanagh, J. T. Triffitt, M. W. Lundy, R. J. Phipps, B. L. Barnett, F. P. Coxon, M. J. Rogers, N. B. Watts, F. H. Ebetino, Ann. NY. Acad. Sci. Skeletal Biology and Medicine, Pt B 2007, 1117, 209-257.
[25] A. J. Roelofs, C. A. Stewart, S. T. Sun, K. M. Blazewska, B. A. Kashemirov, C. E. McKenna, R. G. G. Russell, M. J. Rogers, M. W. Lundy, F. H. Ebetino, F. P. Coxon, J. Bone Miner. Res. 2012, 27, 835-847.
[26] a) M. S. Marma, Z. D. Xia, C. Stewart, F. Coxon, J. E. Dunford, R. Baron, B. A. Kashemirovli, F. H. Ebetino, J. T. Triffitt, R. G. G. Russell, C. E. McKenna, J. Med. Chem. 2007, 50, 5967-5975; b) C. E. McKenna, B. A. Kashemirov, K. M. Blazewska, I. Mallard-Favier, C. A. Stewart, J. Rojas, M. W. Lundy, F. H. Ebetino, R. A. Baron, J. E. Dunford, M. L. Kirsten, M. C. Seabra, J. L. Bala, M. S. Marma, M. J. Rogers, F. P. Coxon, J. Med. Chem. 2010, 53, 3454-3464.
[27] A. J. Roelofs, F. P. Coxon, F. H. Ebetino, M. W. Lundy, Z. J. Henneman, G. H. Nancollas, S. Sun, K. M. Blazewska, J. L. Bala, B. A. Kashemirov, A. B. Khalid, C. E. McKenna, M. J. Rogers, J. Bone Miner. Res. 2010, 25, 606-616.
[28] a) J. Turek, F. H. Ebetino, M. W. Lundy, S. T. Sun, B. A. Kashemirov, C. E. McKenna, M. A. Gallant, L. I. Plotkin, T. Bellido, X. C. Duan, J. T. Triffitt, R. G. G. Russell, D. B. Burr, M. R. Allen, Calcif Tissue Int. 2012, 90, 202-210; b) J. A. Vermeer, I. D. Jansen, M. Marthi, F. P. Coxon, C. E. McKenna, S. Sun, T. J. de Vries, V. Everts, Bone 2013, 57, 242-251; c) A. Verhulst, S. Sun, C. E. McKenna, P. C. D'Haese, Plos ONE 2015, 10, e0121861.
[29] Kolb, H. C.; Finn, M. G.; Sharpless, K. B., Click chemistry: Diverse chemical function from a few good reactions. Angew Chem Int Ed 2001, 40 (11), 2004-2021.
[30] Bock, V. D.; Hiemstra, H.; van Maarseveen, J. H., Cu—I-catalyzed alkyne-azide “click” cycloadditions from a mechanistic and synthetic perspective. Eur J Org Chem 2006, (1), 51-68.
[31] Meldal, M.; Tornoe, C. W., Cu-catalyzed azide-alkyne cycloaddition. Chem Rev 2008, 108 (8), 2952-3015.
[32] Rostovtsev, V. V.; Green, L. G.; Fokin, V. V.; Sharpless, K. B., A stepwise Huisgen cycloaddition process: Copper(I)-catalyzed regioselective “ligation” of azides and terminal alkynes. Angew Chem Int Ed 2002, 41 (14), 2596-2599.
[33] Tornoe, C. W.; Christensen, C.; Meldal, M., Peptidotriazoles on solid phase: [1,2,3]-triazoles by regiospecific copper(I)-catalyzed 1,3-dipolar cycloadditions of terminal alkynes to azides. J Org Chem 2002, 67 (9), 3057-3064.
[34] Kolb, H. C.; Sharpless, K. B., The growing impact of click chemistry on drug discovery. Drug Discov Today 2003, 8 (24), 1128-1137.
[35] Lutz, J. F., 1,3-dipolar cycloadditions of azides and alkynes: A universal ligation tool in polymer and materials science. Angew Chem Int Edit 2007, 46 (7), 1018-1025.
[36] Binder, W. H.; Kluger, C., Azide/alkyne-“click” reactions: Applications in material science and organic synthesis. Curr Org Chem 2006, 10 (14), 1791-1815.
[37] Seo, T. S.; Li, Z. M.; Ruparel, H.; Ju, J. Y., Click chemistry to construct fluorescent oligonucleotides for DNA sequencing. J Org Chem 2003, 68 (2), 609-612.
[38] Moses, J. E.; Moorhouse, A. D., The growing applications of click chemistry. Chem Soc Rev 2007, 36 (8), 1249-1262.
[39] Presolski, S. I.; Hong, V. P.; Finn, M. G., Copper-Catalyzed Azide-Alkyne Click Chemistry for Bioconjugation. Curr Protoc Chem Biol 2011, 3 (4), 153-162.
[40] Skarpos, H.; Osipov, S. N.; Vorob'eva, D. V.; Odinets, I. L.; Lork, E.; Roschenthaler, G. V., Synthesis of functionalized bisphosphonates via click chemistry. Org Biomol Chem 2007, 5 (15), 2361-2367.
[41] Gajda, A.; Gajda, T., Synthesis and reactivity of azidoalkylphosphonates, -phosphinates and -phosphine oxides. Curr Org Chem 2007, 11 (18), 1652-1668.
[42] Zhou, X.; Hartman, S. V.; Born, E. J.; Smits, J. P.; Holstein, S. A.; Wiemer, D. F., Triazole-based inhibitors of geranylgeranyltransferase II. Bioorg Med Chem Lett 2013, 23 (3), 764-766.
[43] Guenin, E.; Hardouin, J.; Lalatonne, Y.; Motte, L., Bivalent alkyne-bisphosphonate as clickable and solid anchor to elaborate multifunctional iron oxide nanoparticles with microwave enhancement. J Nanopart Res 2012, 14 (7).
[44] Chamberlain, B. T.; Upton, T. G.; Kashemirov, B. A.; McKenna, C. E., alpha-Azido Bisphosphonates: Synthesis and Nucleotide Analogues. J Org Chem 2011, 76 (12), 5132-5136.
[45] McPherson, J. C.; Runner, R.; Buxton, T. B.; Hartmann, J. F.; Farcasiu, D.; Bereczki, I.; Roth, E.; Tollas, S.; Ostorhazi, E.; Rozgonyi, F.; Herczegh, P., Synthesis of osteotropic hydroxybisphosphonate derivatives of fluoroquinolone antibacterials. Eur J Med Chem 2012, 47, 615-618.
[46] Chen, X. L.; Li, X.; Yuan, J. W.; Qu, L. B.; Wang, S. H.; Shi, H. Y.; Tang, Y. C.; Duan, L. K., Simple, efficient one-pot method for synthesis of novel N-attached 1,2,3-triazole containing bisphosphonates. Tetrahedron 2013, 69 (20), 4047-4052.
[47] Ferrer-Casal, A.; Barboza, A. P.; Szajnman, S. H.; Rodriguez, J. B., 1, 3-Dipolar Cycloadditions of the Versatile Intermediate Tetraethyl Vinylidenebisphosphonate. Synthesis 2013, 45, 2397-2404.
[48] Liu, X. M.; Thakur, A.; Wang, D., Efficient synthesis of linear multifunctional poly(ethylene glycol) by copper(I)-catalyzed huisgen 1,3-dipolar cycloaddition. Biomacromolecules 2007, 8 (9), 2653-2658.
[49] Liu, X. M.; Lee, H. T.; Reinhardt, R. A.; Marky, L. A.; Wang, D., Novel biomineral-binding cyclodextrins for controlled drug delivery in the oral cavity. J Controlled Release 2007, 122 (1), 54-62.
[50] Hein, C. D.; Liu, X. M.; Chen, F.; Cullen, D. M.; Wang, D., The Synthesis of a Multiblock Osteotropic Polyrotaxane by Copper(I)-Catalyzed Huisgen 1,3-Dipolar Cycloaddition. Macromol Biosci 2010, 10 (12), 1544-1556.
[51] Drago, C.; Arosio, D.; Casagrande, C.; Manzoni, L., Bisphosphonate-functionalized cyclic Arg-Gly-Asp peptidomimetics. Arkivoc 2013, ii (Issue in Honor of Prof Richard R. Schmidt), 185-200.
[52] C. E. McKenna, B. A. Kashemirov, K. M. Blazewska, I. Mallard-Favier, C. A. Stewart, J. Rojas, M. W. Lundy, F. H. Ebetino, R. A. Baron, J. E. Dunford, M. L. Kirsten, M. C. Seabra, J. L. Bala, M. S. Marma, M. J. Rogers, F. P. Coxon, J. Med. Chem. 2010, 53, 3454-3464.
[53] B. A. Kashemirov, J. L. Bala, X. Chen, F. H. Ebetino, Z. Xia, R. G. G. Russell, F. P. Coxon, A. J. Roelofs, M. J. Rogers, C. E. McKenna, Bioconjugate Chem. 2008, 19, 2308-2310.
[54] L. Rocheblave, F. Bihel, C. De Michelis, G. Priem, J. Courcambeck, B. Bonnet, J. C. Chermann, J. L. Kraus, J. Med. Chem. 2002, 45, 3321-3324.
[55] S. S. White, H. T. Li, R. J. Marsh, J. D. Piper, N. D. Leonczek, N. Nicolaou, A. J. Bain, L. M. Ying, D. Klenerman, J. Am. Chem. Soc. 2006, 128, 11423-11432.
[56] B. A. Kashemirov, J. L. Bala, X. Chen, F. H. Ebetino, Z. Xia, R. G. G. Russell, F. P. Coxon, A. J. Roelofs, M. J. Rogers, C. E. McKenna, Bioconjug. Chem. 2008, 19, 2308-2310.
[57] a) I. V. Kutyavin, S. G. Lokhov, I. A. Afonina, R. Dempcy, A. A. Gall, V. V. Gorn, E. Lukhtanov, M. Metcalf, A. Mills, M. W. Reed, S. Sanders, I. Shishkina, N. M. J. Vermeulen, Nucleic Acids Res. 2002, 30, 4952-4959; b) I. Mende, P. Hoffmann, A. Wolf, R. Lutterbuse, E. Kopp, P. A. Baeuerle, A. de Baey, P. Kufer, Int. Immunol. 2005, 17, 539-547; c) C. Lefevre, H. C. Kang, R. P. Haugland, N. Malekzadeh, S. Arttamangkul, R. P. Haugland, Bioconjugate Chem. 1996, 7, 482-489; d) D. Maurel, J. Kniazeff, G. Mathis, E. Trinquet, J. P. Pin, H. Ansanay, Anal. Biochem. 2004, 329, 253-262; e) M. Balaz, M. Sundberg, M. Persson, J. Kvassman, A. Monsson, Biochemistry (Mosc). 2007, 46, 7233-7251; f) Li-Cor IRDye 800CW, SE sectroscopic property, on the World Wide Web at licor.com/bio/products/reagents/irdye/800cw/nhs_ester (accessed October, 2014); g) Lumiprobe Sulfo-Cy5, SE sectroscopic property, on the World Wide Web at lumiprobe.com/p/sulfo-cy5-nhs-ester (accessed October, 2014).

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A toolkit for use in bone tissue, comprising a plurality of N-heterocyclic bisphosphonates, or phosphonocarboxylate analogues thereof, linked to imaging tags, wherein the imaging tag-linked N-heterocyclic bisphosphonates, or phosphonocarboxylate analogues thereof, exhibit preselected physical, optical and biological characteristics.

2. The toolkit of claim 1, comprising the imaging tag-linked N-heterocyclic bisphosphonates, or the phosphonocarboxylate analogues thereof, selected from the group consisting of 5 (6)-FAM-RIS (7a1), 5-FAM-RIS (7a2), 6-FAM-RIS (7a3), 5 (6)-RhR-RIS (7a4), 5 (6)-ROX-RIS (7a5), AF647-RIS (7a6), 5 (6)-FAM-RISPC (7b1), 5 (6)-RhR-RISPC (7b2), 5 (6)-ROX-RISPC (7b3), AF647-RISPC (7b4), 5 (6)-FAM-dRIS (7c1), 5 (6)-RhR-dRIS (7c2), 5-FAM-ZOL (7d1), 6-FAM-ZOL (7d2), AF647-ZOL (7d3), 800 CW-ZOL (7d4), Sulfo-Cy5-ZOL (7d5), 5-FAM-MIN (7e1), 6-FAM-MIN (7e2), 5-FAM-MINPC (7f1), and 6-FAM-MINPC (7f2), in Table 1.

3. A method for analyzing bone, bone metabolism, bone interaction with drugs, BP dosing to bone, or BP distribution within bone, comprising exposing a bone to the imaging tag-linked N-heterocyclic bisphosphonates, or the phosphonocarboxylate analogues thereof, of the toolkit of claim 1.

4. The method of claim 3, wherein the bone is external to a subject's body.

5. A method for treating a bone or bone-related disease in a subject in need thereof, comprising treating the subject with one or more of the imaging tag-linked N-heterocyclic bisphosphonates, or the phosphonocarboxylate analogues thereof, of the toolkit of claim 1, and
visualizing the one or more bisphosphonates, or the phosphonocarboxylate analogues thereof, in the subject by in situ fluorescence.

6. The toolkit of claim 1, for treating a bone or bone-related disease in a subject in need thereof with one or more of the imaging tag-linked N-heterocyclic bisphosphonates, or the phosphonocarboxylate analogues thereof, and visualizing the one or more bisphosphonates, or the phosphonocarboxylate analogues thereof, in the subject by in situ fluorescence.

7. A method of preparing a kit (toolkit) for use in bone tissue, comprising combining a plurality of N-heterocyclic bisphosphonates, or phosphonocarboxylate analogues thereof, with activated imaging tags so as to form a toolkit of imaging tag-linked N-heterocyclic bisphosphonates, or phosphonocarboxylate analogues thereof, that exhibit selected physical, optical and biological characteristics,
wherein the imaging tags are linked to the N-heterocyclic bisphosphonates, or the phosphonocarboxylate analogues thereof, by reacting activated imaging tags to ammonolized halogen-containing N-heterocyclic bisphosphonates, or phosphonocarboxylate analogues thereof, and to amino-group containing N-heterocyclic bisphosphonates, or phosphonocarboxylate analogues thereof.

8. The method of claim 7, wherein the imaging tag-linked N-heterocyclic bisphosphonates, or the phosphonocarboxylate analogues thereof, are selected from the group consisting of 5 (6)-FAM-RIS (7a1), 5-FAM-RIS (7a2), 6-FAM-RIS (7a3), 5 (6)-RhR-RIS (7a4), 5 (6)-ROX-RIS (7a5), AF647-RIS (7a6), 5 (6)-FAM-RISPC (7b1), 5 (6)-RhR-RISPC (7b2), 5 (6)-ROX-RISPC (7b3), AF647-RISPC (7b4), 5 (6)-FAM-dRIS (7c1), 5 (6)-RhR-dRIS (7c2), 5-FAM-ZOL (7d1), 6-FAM-ZOL (7d2), AF647-ZOL (7d3), 800 CW-ZOL (7d4), Sulfo-Cy5-ZOL (7d5), 5-FAM-MIN (7e1), 6-FAM-MIN (7e2), 5-FAM-MINPC (7f1), and 6-FAM-MINPC (712), in Table 1.

9. A method of preparing a modified N-heterocyclic bisphosphonate, or a phosphonocarboxylate analogue thereof, comprising
reacting an N-heterocyclic bisphosphonate, or a phosphonocarboxylate analogue thereof, with a haloepoxide to produce a halogen-containing N-heterocyclic bisphosphonate, or a phosphonocarboxylate analogue thereof, and
converting the halogen-containing N-heterocyclic bisphosphonate, or a phosphonocarboxylate analogue thereof, to an amino group-containing N-heterocyclic bisphosphonate, or a phosphonocarboxylate analogue thereof.

10. The method of claim 9, wherein:
a) the N-heterocyclic bisphosphonate, or the phosphonocarboxylate analogue thereof, has the formula: 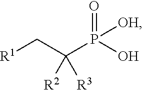
b) the halogen-containing N-heterocyclic bisphosphonate, or the phosphonocarboxylate analogue thereof, has the formula: 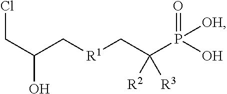
or
c) the amino group-containing N-heterocyclic bisphosphonate, or the phosphonocarboxylate analogue thereof, has the formula: 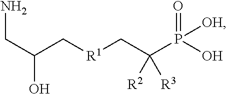
wherein R¹ is an imidazole, a pyridine, or an imidazo[3,2-a]pyridine, R² is H or OH; and R³ is P(O)(OH)₂ or C(O)OH.

11. The method of claim 10, wherein the imidazole is 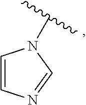 [SMILES: *n1ccnc1]
or an analogue thereof,
the pyridine is 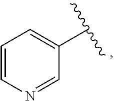 [SMILES: *c1cccnc1]
or an analogue thereof,
and the imidazo[3,2-a]pyridine is 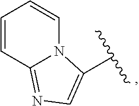 [SMILES: *c1cnc2ccccn12]
or an analogue thereof.

12. The method of claim 9, wherein the haloepoxide is epichlorohydrin.

13. The method of claim 9, further comprising reacting an amino group of the amino group-containing N-heterocyclic bisphosphonate, or the phosphonocarboxylate analogue thereof, with an imaging tag.

14. The method of claim 13, wherein the imaging tag comprises a fluorescent dye.

15. The method of claim 14, wherein the imaging tag comprises an activated succinimidyl ester-containing fluorescent dye for reaction with the amino group.

16. The method of claim 13, wherein the reacting of the amino group with the imaging tag produces an N-heterocyclic bisphosphonate, or the phosphonocarboxylate analogue thereof, of the formula: 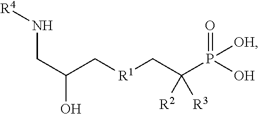
wherein R¹ is an imidazole, a pyridine, or an imidazo[3,2-a]pyridine, R² is H or OH; R³ is P(O)(OH)₂ or C(O)OH; and R⁴ comprises a fluorescent dye.

17. The method of claim 16, wherein the fluorescent dye is 5 (6)-Carboxyfluorescein, Rhodamine Red X, X-Rhodamine, Alexa Fluor 647, IRDye 800 CW, or Sulfo-Cy5.

18. The method of claim 17, wherein the yield of the fluorescently tagged amino group-containing N-heterocyclic bisphosphonate, or a phosphonocarboxylate analogue thereof, is 50%-77%.

19. A method of preparing a modified N-heterocyclic bisphosphonate or a phosphonocarboxylate analogue thereof, comprising
reacting an epoxide having a protected amino group with an ester-protected N-heterocylic bisphosphonate, or a phosphonocarboxylate analogue thereof, to produce a protected amino group- and hydroxyl group-containing ester-protected N-heterocylic bisphosphonate, or a phosphonocarboxylate analogue thereof,
reacting the protected amino group- and hydroxyl group-containing ester-protected N-heterocylic bisphosphonate, or the phosphonocarboxylate analogue thereof, with a sulfonyl halide to produce a sulfonylated and protected amino group-containing ester-protected N-heterocylic bisphosphonate, or a phosphonocarboxylate analogue thereof,
reacting the sulfonylated and protected amino group-containing ester-protected N-heterocylic bisphosphonate, or the phosphonocarboxylate analogue thereof, with an azide to produce a protected amino group- and azido-containing ester-protected N-heterocylic bisphosphonate, or a phosphonocarboxylate analogue thereof, and
deprotecting the protected amino group- and azido-containing ester-protected N-heterocylic bisphosphonate, or the phosphonocarboxylate analogue thereof, to produce an N-heterocylic bisphosphonate comprising an azido group and an amino group, or a phosphonocarboxylate analogue thereof.

20. The method of claim 19, wherein
a) the ester-protected N-heterocylic bisphosphonate, or phosphonocarboxylate analogue thereof, has the formula: 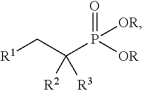
b) the protected amino group- and hydroxyl group-containing ester-protected N-heterocylic bisphosphonate, or phosphonocarboxylate analogue thereof, has the formula: 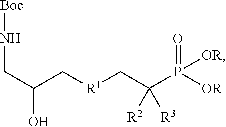
c) the sulfonylated and protected amino group-containing ester-protected N-heterocylic bisphosphonate, or phosphonocarboxylate analogue thereof, has the formula: 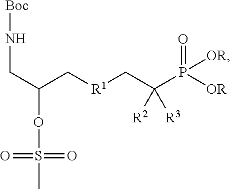
d) the protected amino group- and azido-containing ester-protected N-heterocylic bisphosphonate, or phosphonocarboxylate analogue thereof, has the formula: 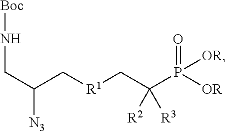
or
e) the N-heterocylic bisphosphonate comprising an azido group and an amino group, or phosphonocarboxylate analogue thereof, has the formula: 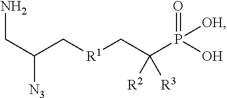
wherein R=Me, Et, Pr or iPr; R¹ is an imidazole, a pyridine, or an imidazo[3,2-a]pyridine; R² is H or OH; and R³ is P(O)(OH)₂ or C(O)OH.

21. The method of claim 20, wherein the imidazole is 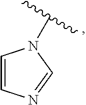 [SMILES: *n1ccnc1]
or an analogue thereof,
the pyridine is 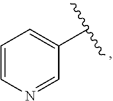 [SMILES: *c1cccnc1]
or an analogue thereof,
and the imidazo[3,2-a]pyridine is 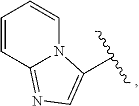 [SMILES: *c1cnc2ccccn12]
or an analogue thereof.

22. The method of claim 19, wherein the expoxide has the formula 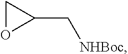 [SMILES: CC(C)(C)OC(=O)NCC1CO1]
the sulfonyl halide is methanesulfonyl chloride, or the azide is NaN₃, or any combination thereof.

23. The method of claim 19, further comprising reacting the azido group or the amino group, or each independently, to a substance of the group consisting of an imaging tag, a drug, a protein, a peptide, an oligonucleotide, a nanoparticle and a polymer.

24. The method of claim 23, wherein the substance comprises an activated succinimidyl ester for reaction with the amino group, or comprises an alkyne for reaction with the azide group.

25. The method of claim 19, wherein the yield of the N-heterocylic bisphosphonate comprising an azido group and an amino group, or a phosphonocarboxylate analogue thereof, is about 28%.

26. A bifunctional N-heterocylic bisphosphonate comprising an azido group and an amino group, or a phosphonocarboxylate analogue thereof, of the formula: 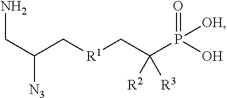
wherein R=Me, Et, Pr or iPr; R¹ is an imidazole, a pyridine, or an imidazo[3,2-a]pyridine; R² is H or OH; and R³ is P(O)(OH)₂ or C(O)OH.

27. The bisphosphonate, or phosphonocarboxylate analogue thereof, of claim 26, wherein the imidazole is 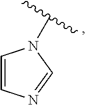 [SMILES: *n1ccnc1]
or an analogue thereof,
the pyridine is 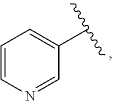 [SMILES: *c1cccnc1]
or an analogue thereof,
and the imidazo[3,2-a]pyridine is 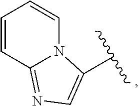 [SMILES: *c1cnc2ccccn12]
or an analogue thereof.

28. A composition comprising an N-heterocylic bisphosphonate, or a phosphonocarboxylate analogue thereof, attached to a substance, wherein the composition has the formula: 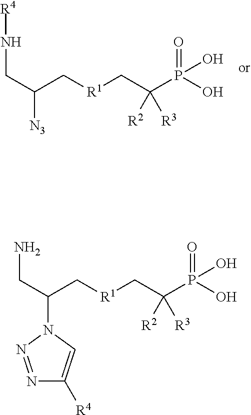
wherein R¹ is an imidazole, a pyridine, or an imidazo[3,2-a]pyridine; R² is H or OH; R³ is P(O)(OH)₂ or C(O)OH, and R⁴ comprises a substance selected from the group consisting of an imaging tag, a drug, a protein, a peptide, an oligonucleotide, a nanoparticle and a polymer.

29. The composition of claim 28, wherein the imaging tag is a fluorescent dye or a fluorescence quencher.

30. The composition of claim 29, wherein the fluorescent dye is 5 (6)-Carboxyfluorescein, Rhodamine Red X, X-Rhodamine, Alexa Fluor 647, IRDye 800 CW, or Sulfo-Cy5; and the fluorescence quencher is Dabcyl, BHQ-1, BHQ-2, BHQ-3, QSY 7, or QSY 9.

31. A method of preparing a modified N-heterocyclic bisphosphonate, or a phosphonocarboxylate analogue thereof, comprising
reacting an N-heterocyclic bisphosphonate, or a phosphonocarboxylate analogue thereof, with an azido epoxide to produce an azido-containing N-heterocyclic bisphosphonate, or a phosphonocarboxylate analogue thereof.

32. The method of claim 31, wherein the azido epoxide is

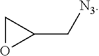

33. The method of claim 31, further comprising reacting an azido group of the azido-containing N-heterocyclic bisphosphonate, or the phosphonocarboxylate analogue thereof, to a substance of the group consisting of an imaging tag, a drug, a protein, a peptide, an oligonucleotide, a nanoparticle and a polymer, wherein the substance contains an alkyne group for reaction with the azido group.
